(19) United States
(12) Patent Application Publication
(10) Pub. No.: US 2025/0131989 A1
(43) Pub. Date: Apr. 24, 2025

(54) LABORATORY CALIBRATION SYSTEM OF MATERIALS FOR USAGE IN SIMULATIONS AND FORECAST OF DISSOLUTION, DISINTEGRATION, DIFFUSION, COMPRESSION, DEFORMATION AND OTHER RELATED MATERIAL PROPERTIES AND CORRESPONDING METHOD THEREOF

(71) Applicant: CINCAP GMBH, Allschwil (CH)

(72) Inventors: Maxim PUCHKOV, Pfeffingen (CH); Guru SIVARAMAN, Affoltern am Albis (CH); Carlo BERTOSSA, Arlesheim (CH)

(73) Assignee: CINCAP GMBH, Allschwil (CH)

(21) Appl. No.: 18/687,503

(22) PCT Filed: Sep. 4, 2021
(86) PCT No.: PCT/EP2021/074430

§ 371 Date: Feb. 28, 2024

(51) Int. Cl. G16C 20/50; G06F 30/20; G16C 20/70; G16C 60/00; G16H 10/20; G16H 20/10

(52) U.S. Cl. CPC G16C 20/50; G06F 30/20; G16C 20/70; G16C 60/00; G16H 10/20; G16H 20/10

(57) ABSTRACT

Proposed is an automated formulation design system and method thereof for determining tablet and capsulation formulations providing a targeted delivery of active pharmaceutical ingredients to a patient with a desired release profile in defined body fluids based on experimental measurements of material attributes for simulated dissolution, disintegration, diffusion, compression, deformation and/or other properties of individual materials or combinations and mixtures thereof. The formulation at least comprises required quantities of the required active pharmaceutical ingredients and excipients providing targeted dissolution and/or solubility factors and/or targeted protection of the active pharmaceutical ingredients during their transport and distribution into interstitial and intracellular fluids.

### FIELD OF THE INVENTION

The present invention relates to instrumentally defining and experimentally measuring of material attributes for usage in simulation systems of dissolution, disintegration, diffusion, compression, deformation, and other related properties for individual materials and their combinations and mixtures. The material attributes are physical or chemical properties of a substance or substances' combination or mixtures which can be measured with a specific instrument, e.g., density, viscosity, Young's modulus, etc. Individual material attributes are converted into a digital representation which may provide a basis for parallel computational simulation systems.

### BACKGROUND OF THE INVENTION

The formulation development is the process by which tablets and capsules are designed to achieve the targeted delivery of medicines to the patient. The component substances of a formulation are the required quantities of active pharmaceutical ingredients (APIs), which provide the medicinal effect, and excipients, which are added to achieve targeted dissolution and solubility or to protect the APIs as they travel through the patient's body to their destinations. The formulation itself begins with the desired physical configuration of the end product (tablet or capsule), including the overall shape and size, as well as the configuration of the APIs and excipients into elements such as granules, layers, and coatings. These are designed to take into account the process of ingestion in the patient's body in order to achieve the effects described above at the right time and place. The second task in formulation development is to determine the appropriate processes, machines, and parameters, from the choices available in commercial pharmaceutical manufacturing, in order to achieve the desired physical configuration, both at a laboratory scale for clinical trials and at a large scale for mass production. The result of a successful formulation is a tablet or capsule with the desired physical configuration that can be manufactured within cost and quality. Since the physical configuration of a manufactured tablet cannot precisely be measured at a reasonable cost, the tablet or capsule is dissolved in the wet lab to determine its release profile—proportion dissolved vs. time—in simulated body fluids. This provides an approximation for the behavior of the tablet or capsule as it is ingested and absorbed by the patient's body. In this manner, the original objective of the formulation—to deliver its components to the body at the right time and place, in the right amount, form, and quality—can be verified.

The challenge facing pharmaceutical scientists today is that formulation is currently done physically in the lab using real substances, processes, and machines. In this sense, designing a tablet or capsule is similar to baking a muffin—and designing a formulation is similar to creating a recipe for a new type of muffin: it can take time and effort over many attempts to get the desired result. Moreover, during an early phase of clinical development, the APIs, which are candidates for the new drug and often new chemical substances, may be prohibitively expensive to produce and may be toxic, requiring costly specialized facilities in which to conduct tests. Each time a new recipe is tried out, it can take several weeks to procure the API, produce the tablet, and measure the resulting release profile in the wet lab. Given this current state of the art, there is a clear need for systems allowing to avoid spending time and money in formulation design for a large number of projects and candidate APIs that are in early stages of development. By their nature, such projects have a low statistical probability of success—formulation work would be wasted effort in projects that subsequently fail for other reasons. Instead, the goal in early stages of clinical development is to filter out unsuccessful projects and APIs as quickly and cheaply as possible. For this reason, Phase 1 and 2 Trials are conducted using primitive service dosage forms—simple capsule shells with the API in powder form poured in to an approximate amount. Formulation work is only undertaken when the API has succeeded in Phase 1 and 2 Trials. Then, a targeted formulation can be designed to make the medicine convenient for the patient to consume, and eventually—if the project succeeds far enough—efficient to produce at large scale. During Phase 1 and 2 Trials, the service dosage form is provided to test subjects.

For the purpose of FDA (Food and Drug Administration) approvals, the release profile of the delivery form is measured and recorded at each Phase. If an API succeeds in Phase 1 Trials, its release profile becomes a mandatory benchmark for each subsequent phase of development. As consequences of today's approach, there are various disadvantages: (1) API Failure due to Service Form: Because of the imprecise and inconsistent nature in which service dosage forms are prepared, there is a substantial risk of candidates failing in Phase I and II Trials, even though the API may have had a real chance to provide therapeutic value given the right delivery profile; (2) Formulation for Bioequivalence—Failure or Delay: If a project and specific API candidate does succeed in Phase I and II Trials, then their chances of eventually making it to market are much higher. The number of projects in the company as a whole that survive this far is also significantly smaller. Therefore, at this point, it becomes feasible to spend the time and effort required to design a targeted tablet or capsule formulation. However, in order to receive FDA approval, the formulation that is designed at this stage must demonstrate bioequivalence with all release profiles recorded during earlier stages of development. This requirement severely constrains the formulation process. Since the earlier release profiles were measured for service dosage forms that were produced imprecisely and inconsistently, the formulation scientist now has to achieve, with their design, an arbitrary and suboptimal release profile, which can be much more difficult than if they were designing a formulation from scratch. Achieving a bioequivalence can take up to a year and several million dollars in time and cost. If bioequivalence cannot be achieved, then the candidate must be discarded; (3) Formulation for Scale-Up—Failure or Delay: If a project survives bioequivalence and Phase Ill Trials, the focus shifts toward FDA approval for market entry and scale-up of the tablet or capsule design from lab equipment to mass production equipment. The formulation typically needs to be re-worked or optimized in order for large-scale machines to produce the desired physical configuration. At this stage, formulation work is much more expensive, as the cost of trying out a new recipe can involve machines costing millions of dollars per day to operate, reset and clean, as well as batches of components hundreds of kilos in size costing millions of dollars. As before, a formulation change at this stage must demonstrate bioequivalence with all release profiles recorded during earlier stages of development, severely constraining the options available to “rescue and repair” a design that doesn't work. If a formulation cannot be repaired for scale up, this may result in a project being cancelled just before market entry.

From technical point of view, the discussed disadvantages are important. By their nature, projects requiring formulation work during bioequivalence and scale-up are the very projects that have the greatest chance of success, and are therefore under enormous pressure to make it to market fast. The longer it takes to achieve a bioequivalent formulation, or to “rescue and repair” a formulation for scale-up, the less time is available for the product to be sold exclusively on the market and to recoup its investment. Past a certain point, this window of profit will close and the project will have to be written off. This can happen at the last minute, sometimes after more than ten years of work and hundreds of millions of dollars of investment.

To understand the need for newer systems and methods in development of modern pharmaceutical drug formulations, it is important to understand the main prior art approaches trends of the past. As mentioned above, development of modern pharmaceutical drug formulations is a challenge; it is time and resource expensive. There is a lot of research and engineering carried out to automate and facilitate this work from the instrumental as well as from the modeling and structural predicting point of view. In general, only one out of 10 000 molecules extensively rested in the discovery phase reaches the marker. Between the discovery of an interesting drug substance and the introduction of the final marketed dosage form of the successful drug substance, a complexity of activities and tests are required. Financial expenditure is estimated to be approximately US$1 billion for a company to introduce a new medication to the market. This expenditure also has to cover the costs of the interesting drug substances that failed during development and did not reach patients. This limits companies to reduce the price of their medications on the market in order to be able to continue R and D activities. Thus, if the costs of R and D could be reduced, all healthcare systems could benefit. If time to market could be reduced, then substantial savings could be achieved. Let be assumed that a new, successful medication may contribute US$365 million to the sales volume of a company in its first year on the marker. Thus, each day earlier on the market could mean US$1 million more in sales. In the case of a new approach using e-Development, such improvements are no longer a distant possibility from an industrial point of view.

In the classical approach, a substantial slowdown concerning ‘time to market’ is linked intrinsically co the workflow and sequential activities. As a result of a lack of connectivity, problems often occur at the interfaces between the different departments involved. These different groups in research, early development, pilot plane manufacturing and scale-lip, or large-scale manufacturing, usually have very different cultures. For this reason, a produce manager is required to follow a product from the beginning, starring with R and D through to large-scale manufacturing of the final marketed dosage form. In general, for early tests in the preclinical and first clinical phases, a preliminary ‘service dosage form’ is prepared, usually a hard gelatin capsule formulation. Such a ‘service dosage form’ may be very different from the final marketed form, which is in general ready at the end of clinical phase 2 or at the beginning of clinical phase 3. Such an approach has many risks. The final marketed dosage form must be bioequivalent to its earlier ‘service dosage form’, which was far from optimal because of a lack of development time and resources allocated. Unfortunately, it is not uncommon that expensive ‘repair actions’ are required for development of the final marketed dosage form to achieve bioequivalence. What happens if the early ‘service’ dosage form was far from optimal? In such a case, the final marketed dosage form must be corrected to the quality of the dosage form tested in clinical phase 2. Thus, the market dosage form may not have the optimal bioavailability. Such an effect creates fundamental problems for companies manufacturing generics. To show bioequivalence, these companies may need to reduce the bioavailability of their product to comply with the originator. Unfortunately, only in rare and extreme situations does non-robustness of an early dosage form become evident at an early clinical phase. The fact that early ‘service’ dosage forms are often poor quality is linked to the company having many drug candidate substances in the preclinical phase (approximately 12 or more) in simultaneous development. There are not enough resources and time slots for doing additional expensive laboratory work in order to develop robust formulations for/all the proposed API for the first clinical trials. Thus, it is not surprising that the early dosage forms are far from six-sigma quality. This problem becomes accented because of regulatory issues, as the formulation cannot be changed during the forthcoming clinical trials.

It has been generally accepted that the quality of pharmaceutical products has a quality of approximately two sigma. A ‘snapshot-view’, i.e. single batch assessment of two sigma quality, corresponds to ca. 4.5% defectives. In a sequence of batch productions over a longer period of time, the percentage of defectives of the sum of batches increases to 20%. This higher value is because of drift of individual results as a consequence of measurements over an extensive time period. The resulting poor quality has triggered actions by regulatory authorities and has boosted FDA's PAT (Process Analytical Technology) and Quality by of Design (QbD) initiatives. The PAT initiative has prompted the installation additional in-process control units in manufacturing departments for optimizing quality. Several pharmaceutical companies in Switzerland and Germany have introduced at-line, on-line or in-line near infrared (NIR) spectroscopy control tools for _nearly all process-steps such as raw material identification, blending, drying, and tableting. It is interesting that the PAT initiative did not have the same visible effect on R and D departments with their task to build-in and nor to rest-in the quality, but to implement according to ICH Q8.

The object of pharmaceutical industrial engineering and development is to design a quality product. The design space is proposed by the applicant and is subject to regulatory issues. In this context, the following fundamental key objects arises: Is it possible to increase the quality of a product and to reduce simultaneously the costs in pharmaceutical development? Therefore, the search for new cools and means is mandatory. The application of computer-aided approaches for system-based design of the robust dosage forms may be promising. However, it has to be kept in mind that solid dosage forms have a number of fundamental problems, arising from particulate property of matter. Indeed, the behavior of powders is strictly stochastic, often showing chaotic and critical patterns. Thus, development of new systems and technologies for computer-based methods should be able to deal with non-deterministic and probabilistic performance and confine those within a defined engineering scope.

### SUMMARY OF THE INVENTION

It is an object of the present invention to provide a system, which does not show the disadvantages discussed above. In particular, it is an object of the invention to allow device-based, automated formulation design without having each formulation empirically generated and done physically in the lab using real substances, processes and machines. The system should allow to filter out and reduce the number of APIs, in an early phase of clinical development or even before, since APIs, which are candidates for the new drug and often new chemical substances, are often prohibitively expensive to produce and/or toxic, requiring costly specialized facilities in which to conduct tests. Thus, the system should allow reducing significantly the recipes, which have to be tried out, i.e. the APIs procured, the tablets produced, and the resulting release profiles measured in the wet lab. It is a further object of the invention, to allow a precise and consistent prediction of the nature in which service dosage forms should be prepared, thereby filtering out all or most of the candidates which will fail in Phase I and II Trials, even though the API may have had a real chance to provide therapeutic value given the right delivery profile. The system should allow to determine at an early stage bioequivalence with all release profiles recorded during earlier stages of development, while strongly reducing the number of release profiles. In addition, the system should allow for measuring the earlier release profiles for service dosage forms that were produced precisely and consistently, so that the formulation scientist may provide a design for an optimal release profile, thus, saving time and expenses. Finally, the system should allow optimizing the formulation for large-scale machines to produce the desired physical configuration, at an early time, without the need to re-work and re-optimize the formulation at a later point in time again, i.e. demonstrate again bioequivalence with all release profiles recorded during earlier stages of development for needed reformulations. As can be seen from a prior art overview of existing hardware and/or software-based systems, an integral technical approach is still missing. Different methods used in construction of the expert systems prevent any compatibility between the systems. There is a clear need for a more consistent approach to designing and assessing the performance of a pharmaceutical formulation. One of the candidates for such a role is cellular automata. Thus, a further object is to provide an appropriate system based on cellular automata technology, which does not show the disadvantages of the prior art systems and devices.

According to the present invention, these objects are achieved, particularly, with the features of the independent claims. In addition, further advantageous embodiments can be derived from the dependent claims and the related descriptions.

According to the present invention, the above-mentioned objects for an automated formulation design system for determining tablet and capsulation formulations providing a targeted delivery of active pharmaceutical ingredients (API) to a patient with a desired release profile in defined body fluids based on experimental measurements of material attributes for simulated dissolution, disintegration, diffusion, compression, deformation and/or other properties of individual materials or combinations and mixtures thereof are achieved, particularly, in that the formulation at least comprises required quantities of the required active pharmaceutical ingredients (API) and excipients providing targeted dissolution and/or solubility factors and/or targeted protection of the active pharmaceutical ingredients (API) during their transport and distribution into interstitial and intracellular fluids characterized, in that system comprises a first and second task engine, the first task engine capturing a targeted physical configuration of a tablet or capsule as end product comprising at least a shape and/or size and/or configuration of the active pharmaceutical ingredients (API) and/or excipients into elements comprising granules, layers and/or coatings, and the second task engine determining processes, machines and/or operating parameters required to achieve the targeted physical configuration at a laboratory scale clinical trials and/or at large scale for mass production, in that the system comprises discretized voxels in from of a polyhedral meshwork structure, wherein a combination of measured material attributes are mappable onto the discretized voxels of the polyhedral meshwork structure, wherein the polyhedral meshwork structure is defined in an adaptable number of dimensions, each representing an individual material, when mapped onto each face or an edge or a vertex of the meshwork polyhedron, and in that the system comprises a dissolution module simulating the production of the end product by varying the targeted physical configuration of the first task engine, and the parameterized processes and machines of the second task engine, and by generating the effect of variances in component, process and machine parameters and the corresponding release profile of the tablet or capsule as end product. Said individual material can, for example, be represented by means of floating point material attribute functional coefficients, when mapped onto each face or an edge or a vertex of a meshwork polyhedron. Said individual material can also e.g. be represented by means of integer functional coefficients, when mapped onto each face or an edge or a vertex of a meshwork polyhedron, or by means of short word functional coefficients, when mapped onto each face or an edge or a vertex of a meshwork polyhedron, or by means of byte type functional coefficients, when mapped onto each face or an edge or a vertex of a meshwork polyhedron. For said combination, all material attributes from the simulated mixture or mixtures thereof can, for example, be storable by means of the voxel. Said combinations can e.g. be usable in diffusion simulation, or in dissolution simulation, or in disintegration simulations, or in swelling simulation, or in compression simulation, or in post-compressive stress distribution and lamination/capping simulation. The simulation and development of a targeted tablet or capsule formulation achieving a targeted release profile in simulated body fluids comprises the system can, for example, comprise a calibration module calibrating the simulation environment and the system to match real-world behavior of substances and processes, wherein the formulation is developed by means of the calibrated simulation environment. One of the advantages of the present system is to provide an automated, technical solution enabling pharmaceutical scientists or man skilled in the art to design targeted tablet and capsule formulations in a virtual environment. Using the inventive system, the users can select component substances such as APIs and excipients, in the required amounts, and define a desired physical configuration for the tablet or capsule. They can select and configure the manufacturing processes, machines and parameters required to achieve the desired configuration. Using the measured characteristics of the component substances, processes and machines, the system is able to simulate the physical configuration that will result, as well its expected release profile (dissolution) in simulated body fluids. Because this is achieved in a virtual environment provided by the system, the system is not subject to physical processes to produce substances, manufacture tablets or conduct physical analysis work in the wet lab. As a result, experimenting with formulations—which can take weeks and cost money for substances, equipment and personnel for a single test—can be done in minutes using the present system. Moreover, with every project, the system allows to add to its growing knowledge base of substances, processes and machines. The present system can be used at different stages in the clinical development process with corresponding degrees of potential benefit. Inter alia, the present invention has the following further advantages and desired technical effects: (i) “Rescue and Repair” during scale-up providing reduced risk of failure and accelerated time to market: During the late stage of production scale-up, the present system is able to provide faster iterations in the rescue and repair process. Although the design constraints from Phases 1 to 3 can be severely restricting, if a solution can be found, it is still possible to find it faster with the present system. This can mean the difference between saving a project and writing it off. To the extent that a solution can be found faster with the present system, it is also possible to rescue a longer window of market exclusivity for the product; (ii) “Rescue and Repair” during Bioequivalence providing reduced risk of failure and accelerated time to market: During the bioequivalence phase, designing formulations with the present system is similarly constrained by the suboptimal release profiles benchmarked in Phases 1 and 2 using the service dosage form. Nevertheless, the invention enables much faster design and prototyping of formulations to reduce the time spent in achieving bioequivalence and increase its chances of success. Formulations designed with the present invention also have better optimized windows of tolerance, which reduce the risk of unsolvable issues emerging later during production scale-up; (iii) “Formulation First” for Maximum Benefit: One of the most effective use of inventive system is when the invention is used to design formulations for projects before clinical trials begin. Naturally, in this paradigm, formulation work is also done for projects that will subsequently fail. However, if the time and cost spent in formulation using the present system is low enough per project, the benefits across the organization can more than outweigh the additional cost: (a) Reduced risk of failure: By using the invention before Phase I trials, the formulation can be designed within the largest design space, as there are no limiting constraints from previous phases of development, and can therefore be highly precise and optimized, offering the greatest chance of success for API candidates; (b) Accelerated time to market: Since the formulation is done before trials begin, the bioequivalence phase is no longer needed. If the initial formulation is also verified with probable production processes and equipment, the scale-up phase can also be reduced or eliminated; (c) Stronger IP protection: If desired, multiple formulations can be designed to achieve the same delivery profile. These can be registered individually over time in order to strengthen the patent protection for a potential blockbuster; (d) Combination drugs: The inventive system can be used to develop formulations that are unfeasible or uneconomical by traditional methods, such as combination drugs; (e) FDA (Food and Drug Administration) compliance and age-appropriate formulation: Alternately, the system allows to provide multiple formulations designed to meet different user needs, for example those of children vs. adults, thus fulfilling the FDA objectives for age-appropriate formulation; (f) FDA compliance and Quality By Design (QBD) and Process Analytical Technology (PAT): As the present system allows to design and model not only the dissolution but also the fabrication of tablets and capsules, it is possible to simulate the effect of variances in component, process and machine parameters on the physical configuration and consequently on the release profile of a tablet. In other words, process tolerances for all aspects of production can be identified. This provides a solution for fulfilling the QBD and PAT objectives mandated by the FDA that is unparalleled in its cost efficiency for the producer and effectiveness in protecting the consumer. Thus, it is also an advantage of the present invention, that the present inventive system allows to support the formulation design process in the same way the pharmaceutical scientist works, however, automated and with high efficiency. Thus, modeling of powder flow, drug dissolution behavior, compaction, and disintegration of solid dosage for medicaments is predicted to solve the aforementioned tasks. The invention provides a cellular automata with a single technical approach with a single system and method for automated predicting and modeling of all typical unit operations encountered in the standard course of the dosage from development. Finally, the provided inventive system according to the invention allows for a first time a combined predictive parameter generation and parameter processing structure to connect granular strength, applied tableting stress, dissolution and disintegration effects under unified technical structure and approach based on the cellular automata. The invention successfully incorporates the relationship between material attributes and process parameters of the tablet and the drug release. In this approach, it is assumed that any highly heterogeneous system such as a complex tablet formulation can be split into a numerous amount of individual cells with autonomous algorithms governing the state changes. The required simulation structure accuracy is achieved when these cells represent sufficiently small volumes, i.e. yielding several millions of cells to generate, rendering such simulation structure computationally-expensive. In the present invention, the processing speed problem is addressed by fast three dimensional cellular automata structure, which in combination with simple FEM simulation resulted in good agreement with the experimental results.

It is a further advantage of the present invention that the disadvantages of the prior art systems, i.e. the various different types of automated or semi-automated prediction systems developed to predict the relevant parameters of the mechanical and dissolution behavior of particles and tablets including the key factors that will affect the drug product quality. An example of the prior art systems is based on Finite Element Method (FEM), in which the powder is treated as a continuous material, and which can be used to simulate the mechanical behavior such as stress distribution and density distribution of tablets during compaction. Other prior art systems are. For example, based on Discrete Element Method (DEM), which have been applied to characterize the breakage of agglomerates and tablets and have also been applied to predict the drug release of matrix tablets. In addition, DEM and FEM hybrid systems have been developed for particle breakage and compaction simulation. However, ideally, DEM-based systems require large number of virtual particles to take into account the microstructure and component heterogeneity of pharmaceutical tablets. This is a technical problem, since DEM-based systems require high computational power to calculate physical phenomenon for individual particles. Hence, in many cases, DEM-based systems are only applicable to larger particle sizes and less numerous particle systems as compared to those in reality. In addition, numerous input parameters are necessary for these complex systems and the DEM-based systems necessarily need to be validated experimentally, however its experimental measurements are typically difficult. Drug release of tablets is influenced by the solubility of the active pharmaceutical ingredient (API), particle size distribution, granule size and their arrangement. In addition, the particle size distribution can change through the manufacturing processes due to the agglomeration, attrition and fragmentation. In particular, in the tableting process, granules are compacted into a tablet at high compressive stresses, resulting in a decrease in particle size due to deformation and fragmentation. To combine the effects from mechanical compaction and formulation composition on the final properties of pharmaceutical tablets, the present invention integrates cellular automata as technical predicting and modelling technique structure. The system applies the cellular automata structure in order allow parameter prediction and simulation of the drug release of tablets. Three-dimensional cellular structure allows to create the simulation matrices containing several different components with a large number of discrete cubes due to the simplicity of the calculation, as compared to DEM models. For example, the system allows to predict and simulate the disintegration time of tablets, buoyancy and drug release profiles of gastro-retentive floating tablets with the three-dimensional cellular automata structure. The present invention allows to predict and simulate the influence of formulations, tablet porosities and compressive stress on drug release profiles of tablets and establish a new approach elucidating the relationship between material attributes and process parameters of the tablet and the drug release. For this purpose, the three-dimensional cellular automata is realized as a technical structure to simulate the drug release of the fast disintegrating tablets in combination with a FEM/DEM model to account for granular breakage and stress distribution.

As another advantage of the present invention, the invention is able to provide and ensure automated compliance with compliance Parameters such as colors, shapes etc. In this embodiment variant, a TabletDesigner module can be used to develop the custom shapes and volumes of the final medicinal product. This includes, inter alia, the automated handling and triggering of parameters which are compatible with human physiology and are allowed by country or region-specific guidelines. Further limitations on the tablet geometries include shape compatibilities with the production equipment, such as the maximal or minimal size of a tablet. The TabletDesigner module monitors users' activities and corrects the errors online during the design of the shape and volume of the tablet. The tablet design process is split into two states: 1) the 2-dimensional design user interface and 2) the 3-dimensional user interface. In the 2D designer mode, the user, e.g. can be requested to draw a die shape of the product and select the height, caps design, and breaking notch. Every tablet design element has the fundamental limitations dictated by the manufacturability-assessment heuristics programmed in. As an example, a heuristic rule can comprise: IF tablet diameter OR tablet dimensions are greater than 20 mm, THEN the tablet cannot be manufactured by standard rotary tablet press THUS warn the user. In the 3-dimensional modus of operation, the user can generate the final 3d shape of the tablet or a capsule, and the total surface, height, and volume will be calculated and displayed by the software. In addition, the user may select the color of the tablet by applying the region or application-specific color schemes available. The color palettes are designed to contain color allowed for use in humans and are non-toxic. In some territories, specific colors are limited due to religious, economic, or psychological limitations. For example, the medicinal product cannot feature an attractive shape or colors to prevent their misuse by children. Further, the TabletDesigner module can contain heuristics to guide the user and prevent the design of the medicinal product, which cannot be manufactured or used in a specific region or a defined field of application.

As a further advantage of the present invention, the invention is able to provide an automated connection between laboratory and simulation for excipients as well as application programming interfaces (API) using a interface to excipient manufacturers. In this embodiment variant, a material database connector unit interfaces can e.g. provide the connectivity of a F-CAD internal material database with the material producer. The connector's functionality can e.g. include an accessibility interface to the non-SQL document-oriented online database. The material characteristics can e.g. be stored in the JavaScript Object Notation (JSON) object format, i.e., they do not have the limitations of the standard relational databases. The limits are seen as the unavailability of a particular predefined product characteristic, and the supplier may omit the publication of this property in the database. The latter will limit the simulation performance of this material during the in-silico product design; however, it will still allow this component in the simulations. On the contrary, if the manufacturer of the excipient or an active pharmaceutical ingredient has different properties that do not have predesigned placeholders in the document structure, the introduction of those will not cause a system malfunction; those properties will be made available to the end-user of the software. If subsequent versions of the simulation make use of this additional information, then they are already available. The material document can further comprise means for accepting dynamic data, e.g., solubility vs. pH or viscosity vs. concentration. Typically, information provider supplies only measured selected data points; the rest of the inter and extrapolation will be carried out by the F-CAD simulation software using the cubic spline interpolation. An example of the JSON format of the database record for a selected material can e.g. be as follows:

```
{ “uid”:1,
        “name”:“Theophilline”,
        “producer”:“CINCAP”,
        “type”:1,
        “amount”:0,
        “initialAmount”: 0,
        “density”:1453,
        “actualcells”:0,
        “wfraction”:0,
        “vfraction”:0,
        “solubility”:[{“key”:7, “value”:7.36}],
        “molarMass”:180.164,
        “viscosity”:0,
        “viscosityAlpha” : 0,
        “capillaryConstant”:0,
        “contactAngle”: 0,
        “waterBindingRate”:1,
        “LOD”:0.00,
        “diffusivity”:6.16e−10,
        “tension” : 0.0,
        “ssa”:0.1,
        “psize”:0.050,
        “poreSize” : 1.6e−3,
        “porosity”: 0,
        “formFactor”:1,
        “color”:“#ca1f48”
}
```

As another advantage of the present invention, the invention is able to provide an automated connection between lab and simulation for machine process types as well as APIs e.g. for Interface to Machine Manufacturers. The characteristic of the solid pharmaceutical formulations, such as disintegration time, release rate, hardness, etc., depend not only on the composition of the formulation but also on the order and process parameters when constituents were put together. The results may differ tremendously depending on the apparatus model and mode of operation for the same process, e.g., for wet granulation or compaction. The used F-CAD simulation platform offers a built-in compiler language to automate the production of the virtual tablets or granules according to the trait of used machines or processes. For example, the granules in different devices will have different porosity of the granules or roundness of the resulting particles. These changes are introduced by different impeller or chopper geometries and the shape of the granulation vessels or allowed working temperatures. With the help of the built-in programming language, it is possible to exactly match the typical unit operations' behaviors. For example, the invention allows to automatically specify the granulation's typical granulometric composition and final granules' porosity for different constituents to match the specific granulator model. This process requires data obtained by the equipment manufacturer for standard formulations and generalized to other components with the help of the language lexemes. An example of script language can be given as follows:

```
program FCADTEST;
var
  filename,emsg:string;
  ha: TStrings;
  i: integer;
begin
  filename := ‘test.3dpac2’;
  try
    CreateMatrix(154,154,154,0.05);
    MakeRoundTablet(100,50);
    AddComponent(1, ‘ScriptAPI’, 1.2345, clRed);
    Distribute(1, 5, 20);
    Seed(1);
    Grow(1);
    Grow(1);
    Grow(1);
    UpdateStatistics;
    ha := getComponentHistogram(1);
    Writes(‘Length:’);Writel(ha.count);WriteIn;
    for i := 0 to ha.count-1 do begin
    Writes(ha[i]);
    end;
    SaveMatrix(filename);
  except
    emsg:=ExceptionToString(ExceptionType, ExceptionParam);
    writes(emsg);
    //do somethign with the exception message i.e. email it or
    //save to a log etc
  end;
end.
```

Regarding scale up (Buckingham Theorem) and dimensionless parameters, the above-mentioned interface to machine manufacturers can e.g. use the language developed for F-CAD to build a simulation model of the typical behavior of the specified machine and to generalize this behavior on the other components of the formulations. These models can be chained or applied in parallel to come as close as possible to describe the physical process. The mathematical models are based upon dimensionless characteristic numbers or criteria. These numbers allow describing the complex physical or physical-chemical phenomena with relatively simple criteria-based equations with exponents. For example, the Navier-Stokes equation can be represented in the criterial form as follows:

$$\frac{Δ p}{ρ {v}^{2}} = {( \frac{vd ρ}{μ} )}^{m} {( \frac{{v}^{2}}{gd} )}^{n} {( \frac{vt}{d} )}^{p} {( \frac{l}{{d}_{e}} )}^{q}$$

The use of criteria such as Reynolds or Nusselt assumes that those values, once obtained for the laboratory scale experiment, will be valid for further investigations at a larger scale, in theory, at an infinitely large scale. This concept allows for scaling up or down of the descriptive process models used by the F-CAD for simulation of the medicinal product properties at any given scale. The models are built-in with the help of the F-CAD programming language.

In an embodiment variant, the invention acts as an ERP solution for the whole product which allows integration into external manufacturing/supply chain solutions. In this embodiment variant, the TabletDesigner module, Interface to Excipient Manufacturers, Interface to Machine Manufacturers, and Scale-Up are considered as a daisy chain model piggybacking on the F-CAD simulation engine based on the cellular automata (a form of the liner approximations-based numerical method to solve systems of the PDEs) the full functionality of the system is represented in the form of an ERP solution. The F-CAD system is a cloud-based integrator platform offering a user interface to the database access based on different presentations, such as Machine Manufacturers Interface, Excipients Manufacturers Interface, or simulation platform itself, including but not limited to TabletDesigner, Particle Arrangement Module, and the Dissolution Simulations Module. This platform allows Participants in the Pharmaceutical Development, Production and Distribution Value Chain to interact, integrate and orchestrate with other Participants according to their Business Model, either on an End-to-End or Peer-to-Peer basis.

In addition to the system, as described above, and the corresponding method, the present invention also relates to a computer program product that includes computer program code means for controlling one or more processors of the control system such that the control system performs the proposed method; and it relates, in particular, to a computer program product that includes a computer-readable medium that contains the computer program code means for the processors.

### BRIEF DESCRIPTION OF THE DRAWINGS

The present invention will be explained in more detail, by way of example, with reference to the drawings in which:

FIG. 1 shows a block diagram schematically illustrating an automated formulation design system 1 and method according to the invention for determining tablet and capsulation formulations 13 providing a targeted delivery of active pharmaceutical ingredients (API) 111 to a patient with a desired release profile 12 in defined body fluids 121 based on experimental measurements of material attributes 1111/1121 for simulated dissolution 192, disintegration 193, diffusion 191, compression 195, deformation and/or other properties of individual materials 111/112 or combinations and mixtures thereof. The formulation 13 at least comprises required quantities 1115/1125 of the active pharmaceutical ingredients (API) 111 and excipients 112 providing targeted dissolution 1221 and/or solubility 1222 factors and/or targeted protection 1223 of the active pharmaceutical ingredients (API) 111 during their transport and distribution into interstitial 1211 and intracellular 1212 fluids.

FIG. 2 shows a block diagram schematically illustrating the process of formulation, by which tablets and capsules 13 are designed to achieve the targeted delivery 12 of medicines to the patient.

FIG. 3 shows a block diagram schematically illustrating the current state of the art process. The formulation has currently to be done physically in the lab using real substances, processes and machines. In this sense, designing a tablet or capsule is similar to baking a muffin—and designing a formulation is similar to creating a recipe for a new type of muffin: it can take time and effort over many attempts to get the desired result. Moreover, during an early phase of clinical development, the APIs, which are candidates for the new drug and often new chemical substances, may be prohibitively expensive to produce and may be toxic, requiring costly specialized facilities in which to conduct tests. Each time a new recipe is tried out, it can take several weeks to procure the API, produce the tablet, and measure the resulting release profile in the wet lab.

FIG. 4 shows a block diagram schematically illustrating the avoiding of formulation during Phase 1 and 2 Trials in the prior art. Given this current state of the art, pharmaceutical developers are often unwilling to spend time and money in formulation design for the large number of projects and candidate APIs that are in early stages of development. By their nature, such projects have a low statistical probability of success—formulation work would be wasted effort in projects that subsequently fail for other reasons. Instead, the goal in early stages of clinical development is to filter out unsuccessful projects and APIs as quickly and cheaply as possible. For this reason, Phase 1 and 2 Trials are conducted using primitive service dosage forms—simple capsule shells with the API in powder form poured in to an approximate amount. In prior art, formulation work is typically only undertaken when the API has succeeded in Phase 1 and 2 Trials. Then, a targeted formulation can be designed to make the medicine convenient for the patient to consume, and eventually—if the project succeeds far enough—efficient to produce at large scale.

FIG. 5 shows a block diagram schematically illustrating consequences of prior art approach, where during Phase 1 and 2 trials, the service dosage form is provided to test subjects. For the purpose of FDA approvals, the release profile of the delivery form is measured and recorded at each Phase. If an API succeeds in Phase 1 Trials, its release profile becomes a mandatory benchmark for each subsequent phase of development. Because of the imprecise and inconsistent nature in which service dosage forms are prepared, there is a substantial risk of candidates failing in Phase I and II Trials, even though the API may have had a real chance to provide therapeutic value given the right delivery profile. If a project and specific API candidate does succeed in Phase I and II Trials, then their chances of eventually making it to market are much higher. The number of projects in the company as a whole that survive this far is also significantly smaller. Therefore, at this point, it becomes feasible to spend the time and effort required to design a targeted tablet or capsule formulation. However, in order to receive FDA approval, the formulation that is designed at this stage must demonstrate bioequivalence with all release profiles recorded during earlier stages of development. This requirement severely constrains the formulation process. Since the earlier release profiles were measured for service dosage forms that were produced imprecisely and inconsistently, the formulation scientist now has to achieve, with their design, an arbitrary and suboptimal release profile, which can be much more difficult than if they were designing a formulation from scratch. Achieving a bioequivalence can take up to a year and several million dollars in time and cost. If bioequivalence cannot be achieved, then the candidate must be discarded. If a project survives bioequivalence and Phase Ill Trials, the focus shifts toward FDA approval for market entry and scale-up of the tablet or capsule design from lab equipment to mass production equipment. The formulation typically needs to be re-worked or optimized in order for large-scale machines to produce the desired physical configuration. At this stage, formulation work is much more expensive, as the cost of trying out a new recipe can involve machines costing millions of dollars per day to operate, reset and clean, as well as batches of components hundreds of kilos in size costing millions of dollars. As before, a formulation change at this stage must demonstrate bioequivalence with all release profiles recorded during earlier stages of development, severely constraining the options available to “rescue and repair” a design that doesn't work. If a formulation cannot be repaired for scale up, this may result in a project being cancelled just before market entry.

FIG. 6 shows a block diagram schematically illustrating the reason, why the problems as discussed above are important. By their nature, projects requiring formulation work during bioequivalence and scale-up are the very projects that have the greatest chance of success, and are therefore under enormous pressure to make it to market fast. The longer it takes to achieve a bioequivalent formulation, or to “rescue and repair” a formulation for scale-up, the less time is available for the product to be sold exclusively on the market and to recoup its investment. Past a certain point, this window of profit will close and the project will have to be written off. This can happen at the last minute, sometimes after more than ten years of work and hundreds of millions of dollars of investment.

FIG. 7 shows a block diagram schematically illustrating a simulation and prediction system 1 and method, according to the invention. The simulation platform enables the pharmaceutical developers to design targeted tablet and capsule formulations 13 in a virtual environment 151. Using the system 1, the developer can select component substances 111/112 such as APIs 111 and excipients 112, in the required amounts 13114/13124, and define a desired physical configuration 13113/13123 for the tablet or capsule 13. They can select and configure the manufacturing processes 1711, machines 1712 and parameters 1713 required to achieve the desired configuration. Using the measured characteristics of the component substances 111/112, processes 1711, machines 1712 and parameters 1713, F-the system 1 can simulate the physical configuration 13113/13123 that will result, as well its expected release profile 12 (dissolution) in simulated body fluids.

FIG. 8 shows a block diagram schematically illustrating the inventive system 1, being enabled to be used at different stages in the clinical development process with corresponding degrees of potential benefit.

FIG. 9 shows a block diagram schematically illustrating that the present system 1 is able to support the formulation 13 design process in the same way the pharmaceutical developers works.

FIG. 10 shows a block diagram schematically illustrating, that the present system 1 can also be able to build as a fully secure, device-independent cloud solution. The system 1 architecture uses the components and interfaces as shown in FIG. 9.

FIG. 11 shows a block diagram schematically illustrating the system's 1 workflow for solving formulation problems. The system 1 can be used to develop a tablet or capsule formulation 13 that will achieve a desired release profile 12 in simulated body fluids 121. This is done in two phases: first, the system's simulation environment 151 is calibrated to properly model the real-world 152 behavior of substances 11 and processes 171; and second, the calibrated simulation environment 151 is used to develop the desired formulation 13.

FIG. 12 shows a block diagram schematically illustrating a generalized plot of the relation in a form N/N₀=(1−e^{-kt}), where t is the time.

FIG. 13 shows a block diagram schematically illustrating the so-called von Newman and Moore neighborhood structures.

FIG. 14 shows a block diagram schematically illustrating an example of 2-D cellular automata, a solid gets dissolved by liquid.

FIG. 15 shows a block diagram schematically illustrating the evolution of a rule 182 based cellular automata.

FIG. 16 shows a block diagram schematically illustrating the finite-difference 4-dot forward schema providing a solution of the 1 D diffusion relation (i Indicates spatial position and t is time) FIG. 17 shows a block diagram schematically illustrating the graphical representation of rule 182 and Its binary coding.

FIG. 18 shows a block diagram schematically illustrating the solution of the diffusion relation through 1D cellular automata applied rule 182 (10110110). The upper figure depicts 32 iterations of rule 182. The lower figure shows 32 bell-shaped curves of the concentration vs. position and the diagonal line shows the cumulative function of concentration vs. time.

FIG. 19 shows a block diagram schematically illustrating the growth of particles in a simulated tablet: bottom left—seeds; bottom right—grown particles after ninth iteration.

FIG. 20 shows a block diagram schematically illustrating the packing of virtual ‘placeholder’ spheres to find central positions from seeds for further growth of the granules or larger particles of the formulation components.

FIG. 21 shows a block diagram schematically illustrating from left to right the degradation of a porous network (pores depicted as pink) during growth of solid particles (solids are transparent).

FIG. 22 shows a block diagram schematically illustrating a system-generated, i.e. a computer-generated, tablet (left) and real tablet with leached out API (caffeine). (a) Caffeine 500-710 μm, (b) caffeine 250-355 μm, (c) caffeine 1.25-180 μm.

FIG. 23 shows a block diagram schematically illustrating a particle size distribution of Individual particles in a compact with respect to growth Iteration (top left: Initial set, bottom right: after tenth Iteration) FIG. 24 shows a block diagram schematically illustrating a top view of a tablet filled with distributed API cells and surrounded by a steel mantle, as generated by the second task engine 17 (or PAC module).

FIG. 25 shows a block diagram schematically illustrating a side view of a tablet filled with distributed API cells and surrounded by a steel mantle, as generated by second task engine 17 (or PAC module).

FIG. 26 shows a block diagram schematically illustrating iterations of 3-D CA for growing—one particle from a seed (Iteration HV).

FIG. 27 shows a block diagram schematically illustrating a lateral view of a tablet, in particular the lateral cross-section of a tablet.

FIG. 28 shows a block diagram schematically illustrating the generated particle size distribution of the lateral view of a tablet of FIG. 27.

FIG. 29-32 shows a block diagram schematically illustrating a visualization of growth iteration, of a single component, in particular, the growing of micro-crystalline cellulose (MCC)-simulating seeds in a virtual tablet (figures A¹-D¹ and A²-D² of FIG. 29-32) and the associated particle size distributions (figures a-d of FIGS. 29-32).

FIG. 33 shows a block diagram schematically illustrating the basic CA-update rules for pores, as one of the different types of the components, in particular that every liquid cell in the Moore neighborhood of a pore cell subtracts one unit from C1ₚₒᵣₑ value until C1ₚₒᵣₑ=0 and the cell changes its state to that of a liquid cell.

FIG. 34 shows a block diagram schematically illustrating the basic CA-update rules for Active Pharmaceutical Ingredient (API), as one of the different types of the components, in particular that the solubilization property of a non-hydrophobic API cell can be characterized with C1_{API}, no C2 is necessary, wherein C1_{API} defines the number of iterations necessary to change the solid state of the cell to a liquid state.

FIG. 35 shows a block diagram schematically illustrating the basic CA-update rules for hydrophobic API (hAPI), as one of the different types of the components, in particular that the difference to API cells is that a hAPI cell is given a C2_{hAPI}. Hydrophobicity is imitated by giving the pore cells in the Moore neighborhood of an API cell a new C1ₚₒᵣₑ. C1ₚₒᵣₑ is generated by dividing Cl hAPI by C2_{hAPI}.

FIG. 36 shows a block diagram schematically illustrating the basic CA-update rules for Swelling compound (Sw), as one of the different types of the components, in particular that this cell type is used to simulate compounds getting more voluminous when coming into contact with water. Liquid cells in the Moore neighborhood are given (similar to hAPI cell type) a C1, which is calculated by dividing C1_{Sw} by C2_{Sw}. The smaller C2_{Sw} the higher is the expansion capacity of Sw cells.

FIG. 37 shows a block diagram schematically illustrating the basic CA-update rules for Disintegrants (Dis), as one of the different types of the components, in particular that the approach of disintegration simulation, as illustrated here, is an attempt to simulate a quick exposure of API cells to liquid as is the case for disintegrating formulations. The constants of Dis cells are both zero. This results in an immediate change of state after the first iteration

FIG. 38 shows a block diagram schematically illustrating the basic CA-update rules for Fillers absorbing water (e.g. MCC), as one of the different types of the components, in particular that

FIG. 39 shows a block diagram schematically illustrating arbitrary simulated formulation release profile with an enlargement of the first 1.5 minutes. The ‘drug on surface’ effect Is expressed on the fifth minute of the release curve 12.

FIG. 40 shows a block diagram schematically illustrating system 1 generated release curves 12 for Identical formulations 13, identical porosities, masses, and compact volumes. The difference Is the shape of the two compacts: flat round vs. round concave.

FIG. 41 shows a block diagram schematically illustrating the release profiles 12 generated for two different unit operations: direct compaction and wet granulation. Both are simulated curves and the formulation compositions are identical.

FIG. 42 shows a block diagram schematically illustrating the experimental and simulated intrinsic dissolution profile of caffeine.

FIG. 43 shows a block diagram schematically illustrating the experimental and simulated Intrinsic dissolution profile of granulated caffeine.

FIG. 44 shows a block diagram schematically illustrating the experimental and simulated dissolution profile of pure caffeine tablets.

FIG. 45 shows a block diagram schematically illustrating the experimental and simulated dissolution profiles of formulation 1.4.

FIG. 46 shows a block diagram schematically illustrating the experimental and simulated dissolution profiles of formulation with MCC and Ac-Di-Sol.

FIG. 47 shows a block diagram schematically illustrating the experimental and simulated intrinsic dissolution profiles of proquazone.

FIG. 48 shows a block diagram schematically illustrating the experimental and simulated dissolution profiles of pure proquazone tablets.

FIG. 49 shows a block diagram schematically illustrating the discretizer module with a round, flat tablet, wherein the number in ‘Cube size, units’ defines the number of cubes at the edges of the cubic grid around the tablet.

### DETAILED DESCRIPTION OF THE PREFERRED EMBODIMENTS

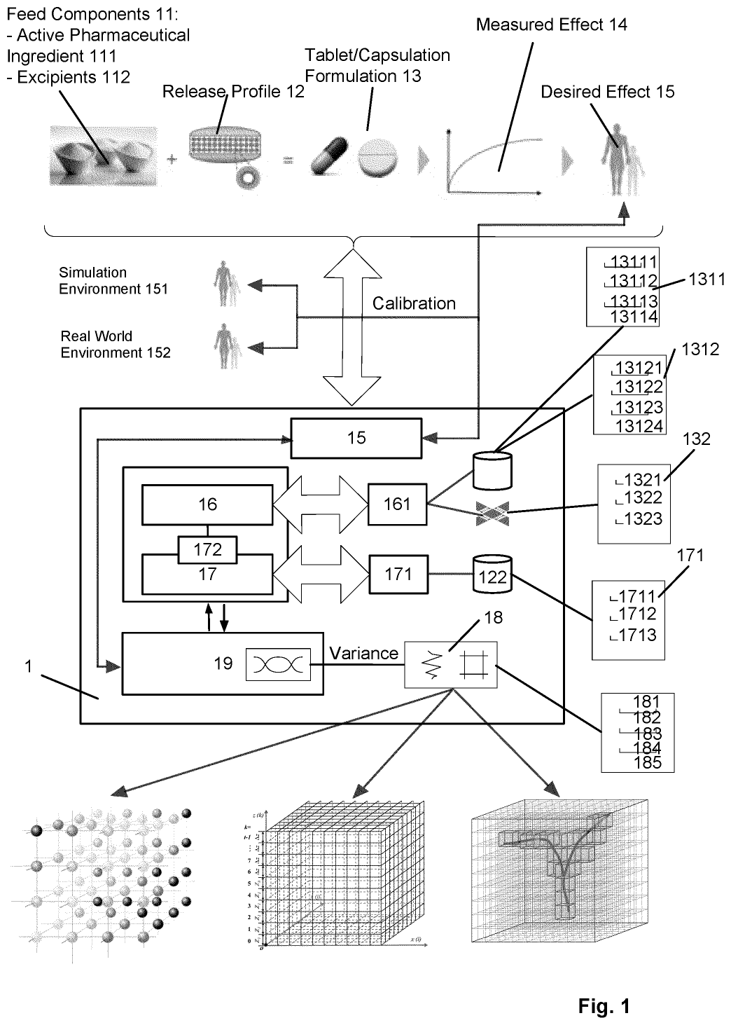

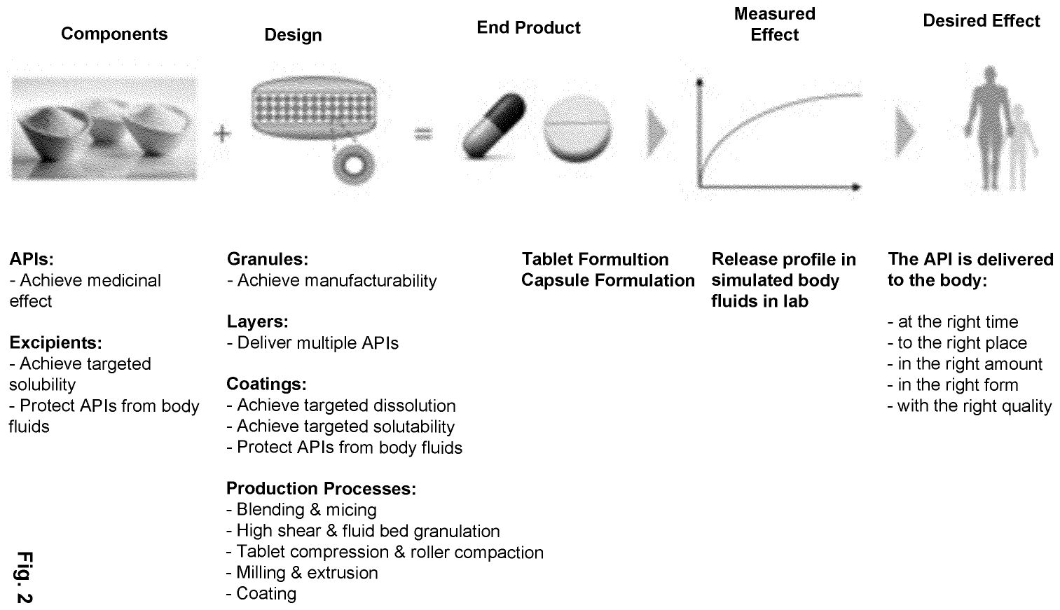

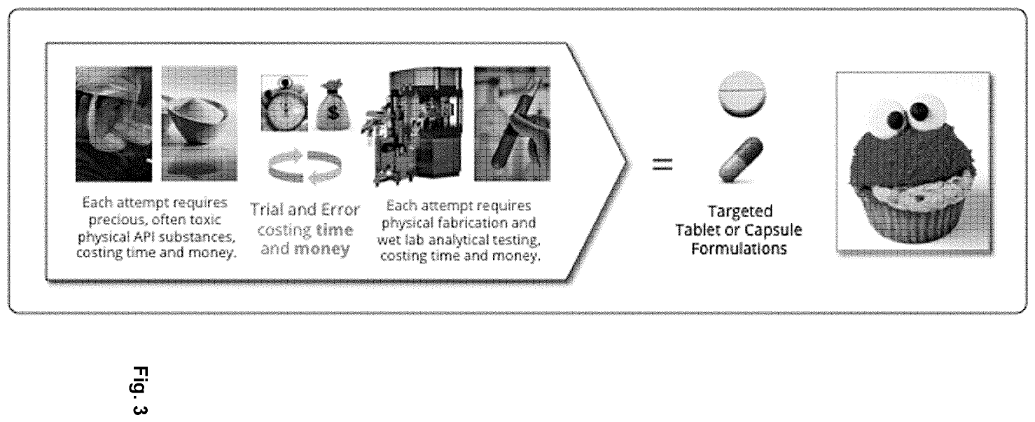

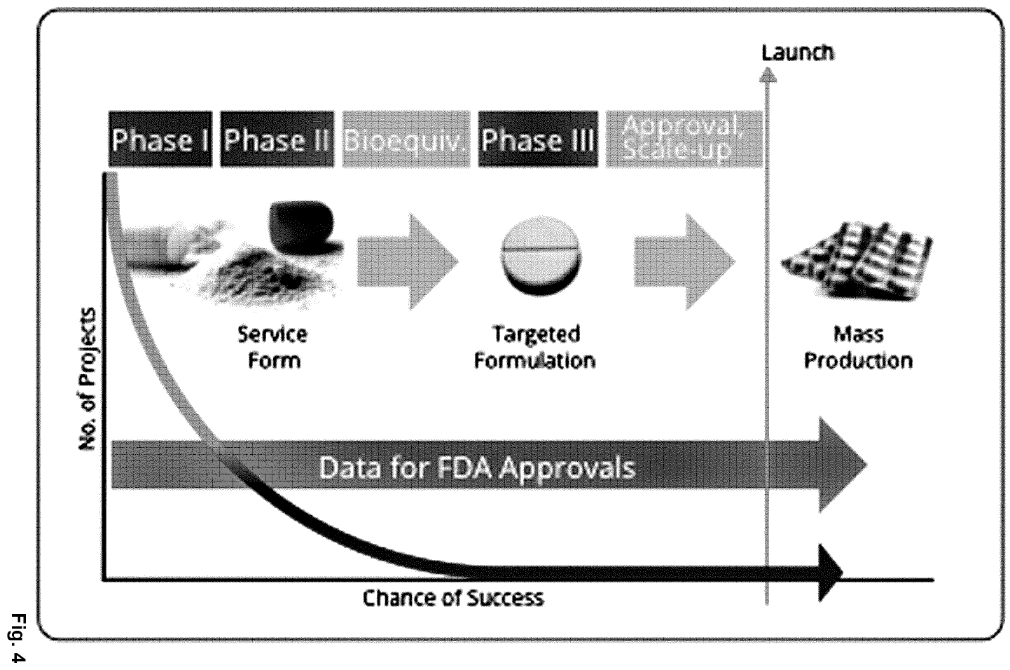

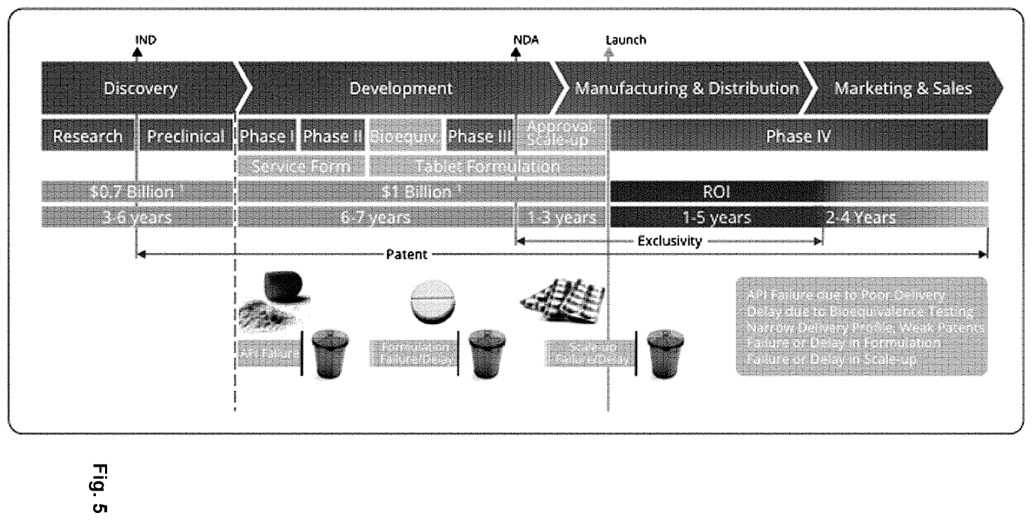

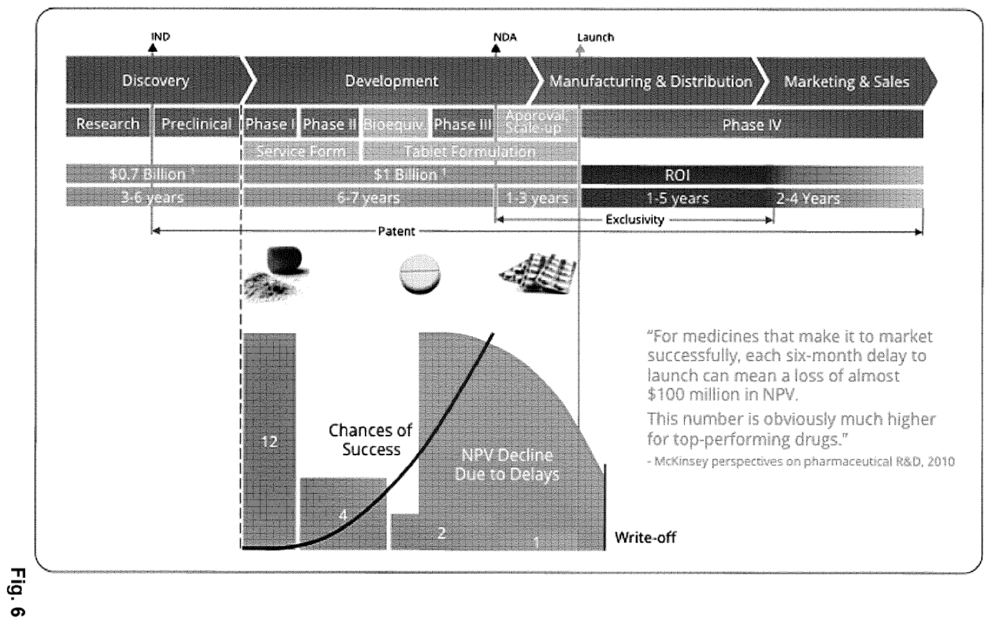

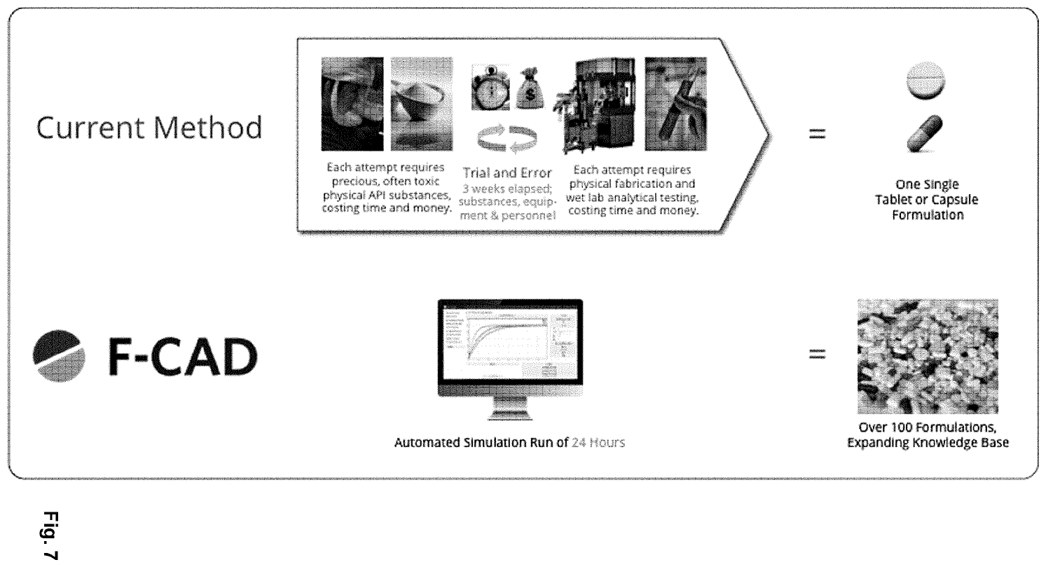

FIG. 1 schematically illustrates an architecture for a possible implementation of an embodiment of the automated formulation design system 1 for determining tablet and capsulation formulations 13 providing a targeted delivery of active pharmaceutical ingredients (API) 111 to a patient with a desired release profile 12 in defined body fluids 121 based on experimental measurements of material attributes 1111/1121 for simulated dissolution 192, disintegration 193, diffusion 191, compression 195, deformation 197 and/or other properties of individual materials 111/112 or combinations and mixtures thereof. The formulation 13 at least comprises required quantities 1115/1125 of the required active pharmaceutical ingredients (API) 111 and excipients 112 providing targeted dissolution 1221 and/or solubility 1222 factors and/or targeted protection 1223 of the active pharmaceutical ingredients (API) 111 during their transport and distribution into interstitial 1211 and intracellular 1212 fluids. Distribution, in the sense of the present application, describes the transfer of a drug from one location to another within the body. Once a drug enters into systemic circulation by absorption or direct administration, it must be distributed into interstitial 1211 and intracellular 1212 fluids. Each organ or tissue may receive different doses of the drug and the drug may remain in the different organs or tissues for a varying amount of time. The distribution of a drug between tissues is dependent on vascular permeability, regional blood flow, cardiac output and perfusion rate of the tissue and the ability of the drug to bind tissue and plasma proteins and its lipid solubility. pH partition plays a major role as well. The drug is easily distributed in highly perfused organs such as the liver, heart and kidney. It is distributed in small quantities through less perfused tissues like muscle, fat and peripheral organs. The drug can be moved from the plasma to the tissue until the equilibrium is established (for unbound drug present in plasma). There may be various factors that affect a drug's distribution throughout an organism. In literature, some of the most important factors are considered to be the following: an organism's physical volume, the removal rate and the degree to which a drug binds with plasma proteins and/or tissues.

System 1 comprises a first and second task engine 16/17. The first task engine 16, in the sense of a tablet and/or capsule designer module captures a targeted physical configuration 161 of a tablet or capsule 13 as end product comprising at least a shape 13111/13121 and/or size 13112/13122 and/or configuration 13113/13123 of the active pharmaceutical ingredients (API) 111 and/or excipients 112 into elements 132 comprising granules 1321, layers 1322 and/or coatings 1323. The first task engine 16 as tablet designer is used to model the desired size and shape of the tablet. The user is able to precisely model shapes of unlimited complexity in three dimensions, and to calculate their overall volume and surface area. This establishes the design space for the subsequent physical design. The design can simply be sent to a 3D printer to produce accurate prototypes. The second task engine 17, as Particle Arrangement and Compaction (PAC) module, determines processes 1711 and/or machines 1712 and/or operating parameters 1713 required to achieve the targeted physical configuration 161 at a laboratory scale clinical trials and/or at large scale for mass production. The PAC module 17 allows the user to define component substances 131/1311/1312 and amounts 13114/13124, and to specify granules 1321, layers 1322 and coatings 1323 as structural elements 131 to achieve a desired physical configuration 13113/13123 for the tablet or capsule 13. At the same time, the user selects and parameterizes the processes 1711 and/or machines 1712 and/or operating parameters that will be used to produce the tablet 13.

The system 1 comprises discretized voxels 181 in from of a polyhedral meshwork structure 18, wherein a combination of measured material attributes 1111/1121 is mapped onto the discretized voxels 181 of the polyhedral meshwork structure 18. The polyhedral meshwork structure 18 is defined in an adaptable number of dimensions, each representing an individual material 11/111/112, when mapped onto each face 183 or an edge 184 or a vertex 185 of the meshwork polyhedron 18. Said individual material 11/111/112 can e.g. be represented by means of material attribute functional coefficients 1112, e.g. by means of floating point material attribute functional coefficients 11121, when mapped onto each face 183 or an edge 184 or a vertex 185 of a meshwork polyhedron 18. Said individual material 11/111/112 can e.g. also be represented by means of integer functional coefficients 11122, or by means of short word functional coefficients 11123, when mapped onto each face 183 or an edge 184 or a vertex 185 of a meshwork polyhedron 18, or by means of byte type functional coefficients 11124, when mapped onto each face 183 or an edge 184 or a vertex 185 of a meshwork polyhedron 18. For said combination, all material attributes 1111/1121 from the simulated mixture or mixtures thereof can e.g. be stored by means of the voxels 181, described above.

The system 1 comprises a dissolution module 19 simulating the production of the end product 13 by varying the targeted physical configuration 161 of the first task engine 16, and the parameterized processes 1711 and/or machines 1712 and/or operating parameters 1713 of the second task engine 17, and by generating the effect of variances in component 131 and/or process 1711 and/or machine 1712 and/or operating 1713 parameters and the corresponding release profile 12 of the tablet or capsule 13 as end product. Thus, the present system 1 is enabled to generate, predict and simulate the production of the tablet 13 according to the ingredients 11 and processes 171 defined above. If these elements are calibrated 15 correctly, the resulting 3D model will be an accurate simulation of the tablet 13 that will be produced if the recipe is applied in the physical lab. I.e., given a 3D model of the expected tablet 13 from the PAC module 17 (second task engine), the dissolution module 19 is then used to simulate the release behavior 12 of this tablet 13 in simulated body fluids 121. Using calibration 15 data for the component substances, the dissolution module 19 accurately predicts the dissolution 1221 of the tablet 12 in simulated body fluids over time. If this release profile 12 meets the original design criteria for the tablet or capsule 13, then the formulation 13 has been successfully achieved. For the simulation and development of a targeted tablet or capsule formulation 13 achieving a targeted release profile 12 in simulated body fluids 121, the system 1 can e.g. comprise a calibration module 15 calibrating the simulation environment 151 and the system to match real-world 152 behavior of substances 131 and processes 171, wherein the formulation 13 is developed by means of the calibrated simulation environment 151.

The inventive system 1 can be used to develop a tablet or capsule formulation 13 that will achieve a desired release profile 12 in simulated body fluids 121. This is done in two phases: first, the system 1 simulation environment 151 is calibrated to properly model the real-world 152 behavior of substances 11/161 and processes 171; and second, the calibrated simulation environment 151 is used to develop the desired formulation 13.

In the calibration phase 15, the first step is to design the tablet's size 13112/13122 and shape 13111/13121 and conceive a physical distribution of its ingredients 11 that is expected to achieve the desired release profile 12. Then, an initial recipe is defined including the necessary processes 1711 and/or machines 1712 and/or operational parameters to produce the tablet 13 in the desired configuration. This first recipe (“Lab Recipe 01”) is then used in the physical lab to produce a first sample tablet 13. The sample is analyzed and its release profile 12 (“Lab result 01”) compared to the desired profile. Typically, the goal is not achieved in the first try. At this point, the “Lab result 01” is recorded and forms the target profile 122 for the calibration 15 of the system 1. “Lab Recipe 01” is translated into the system's machine 1712 and process 1711 and operation parameters 1713 that are then simulated, modified and re-simulated (“F-CAD Recipes 01-03”) until the simulated release profile 123 matches “Lab result 01”. At this point, the simulation environment 151 shows the actual distribution that is resulting from the processes used in “Lab recipe 01”. Once system 1 is calibrated 15, the originally desired release profile 122 forms the target profile 123 for the designer. In the optimization phase, the recipes of the system 1 are adjusted and re-simulated until the simulated release profile 123 matches the desired profile (“F-CAD result 01”). At this point, the recipe of the system 1 can be applied in the physical lab to produce a second sample tablet that is then analyzed. If the results do not sufficiently match, a further optimization can be made within the system 1. The resulting recipe can be produced in the physical lab and will produce the desired release profile 122 when analyzed.

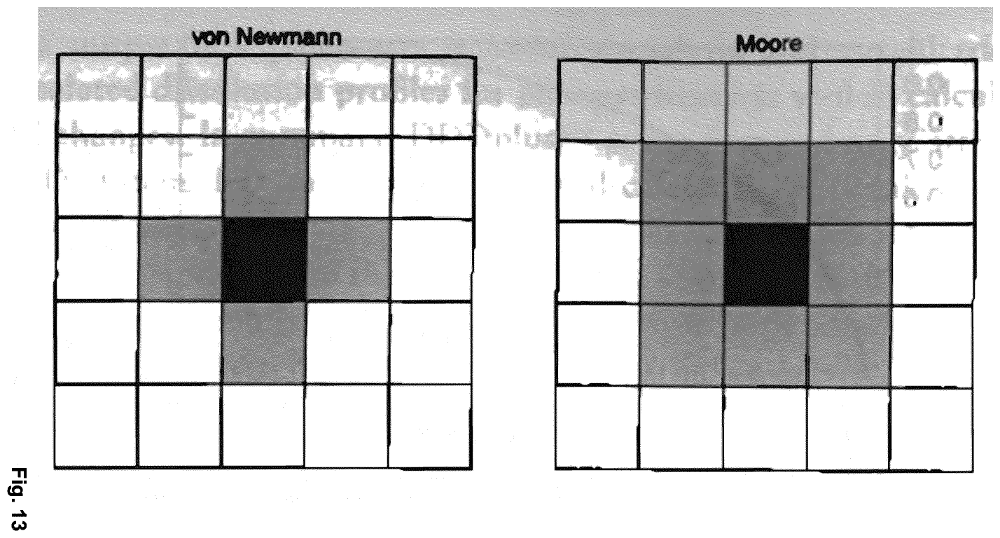

The polyhedral meshwork structure 18 of the system 1 is essential for providing the technical structure of system 1. The discretized voxels 181 and the polyhedral meshwork structure 18 are realized as cellular automata system. Cellular automata can be used for predicting various natural phenomena, as they are often evolutionary, i.e. the development of a system evolves from a certain operation of a previous generation. What makes cellular automata useful for dissolution simulation systems is its ability to take into an account local interactions between domains with different properties. Simple rules can result in complex structures. In this respect, it is important to note that the evolutionary systems are often described by the basic form of the differential equation for evolutionary systems:

$$\frac{}{} = - ,$$

where t is the time, k is the rate constant, and N is the target variable. By separating the variables and integrating this relation, the following form of the exponential functions can be obtained: N=N₀(1−e^{-kt}). As FIG. 12 shows, the chart of the resulting relation represents very closely the typical release profile 12 from tablet formulation 13.

The release speed on the drug from a tablet formulation 13 is dependent on the amount 13114 of the drug released and other associated parameters which regulate the value of the rate constant k. It is to be noted that the release kinetic is an evolutionary system and can be mapped in space and rime. This definition is also common to the cellular automata.

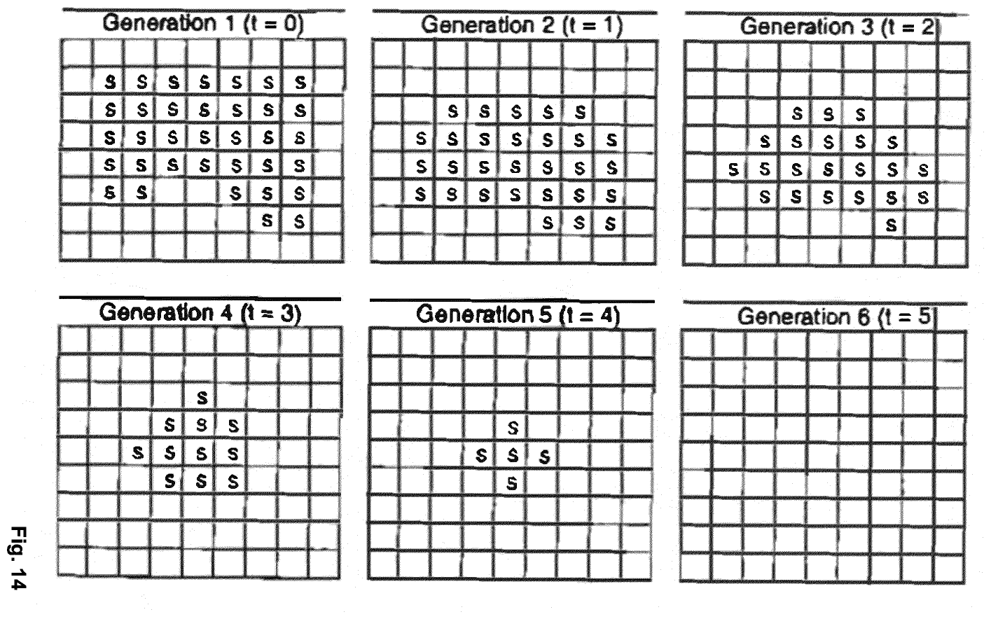

Cellular automata (CA) systems can be defined as follows: Cellular automata are systems based on idealizing physical systems in which space and time are discrete, and the physical quantities take only a finite set of values'. In other words, CA provide discrete models of equally sized cells, arranged in a regular grid, each in one of a finite number of states. After every time seep (so-called iteration or generation), the new state of a cell is determined by its previous state and the states of the neighboring cells. Cells can be classified in different cell types, although on every type a finite number of rules are applied, i.e. every cell of the same type follows the same rules, dependent on the types and states of the neighboring cells. The rules are always applied to all cells, which results in a new generation of cells. The neighborhood can be defined in different ways and there are several types of neighborhood known. Two common neighborhoods, used in the prior art, are shown in FIG. 13, one is the so-called von Newman neighborhood and the other the Moore neighborhood. The blank cells do not affect the stare of the black cells in the next generation, only the grey cells and the state of the black cell itself do. FIG. 14 visualizes 2-D CA system with a simplified example of a solid substance being dissolved in a liquid dissolution medium. The rule set can be as follows: cells of the solid type (s) are changed into a liquid cell (blue) if they are surrounded by four or more liquid cells in the Moore neighborhood. This is a 2-D example of CA with only one rule and two cell types. The more rules, the more types and the more dimensions, the more complex (also natural) behavior can be simulated without changing the complexity of the rules which are kept simple and are always applied on all cells. Technically, the dissolution of a solid in dissolution medium can be expressed using the Noyes-Whitney relation:

$$\frac{}{} ( {}_{} - ) ,$$

Where C is the concentration of the solute at time t, Cₛ is the solubility on the equilibrium at experimental temperature and K is a first-order proportionality constant. The Noyes-Whitney equation in differential form has a form of Fick's second law relation of diffusion and can be represented in the 3-D domain as follows:

$$\frac{Φ}{} = {∇}^{2} Φ ,$$

where ∇² is

$$( \frac{{2}^{}}{{2}^{}} + \frac{{2}^{}}{{2}^{}} + \frac{{2}^{}}{{2}^{}} ) ,$$

[symbol]

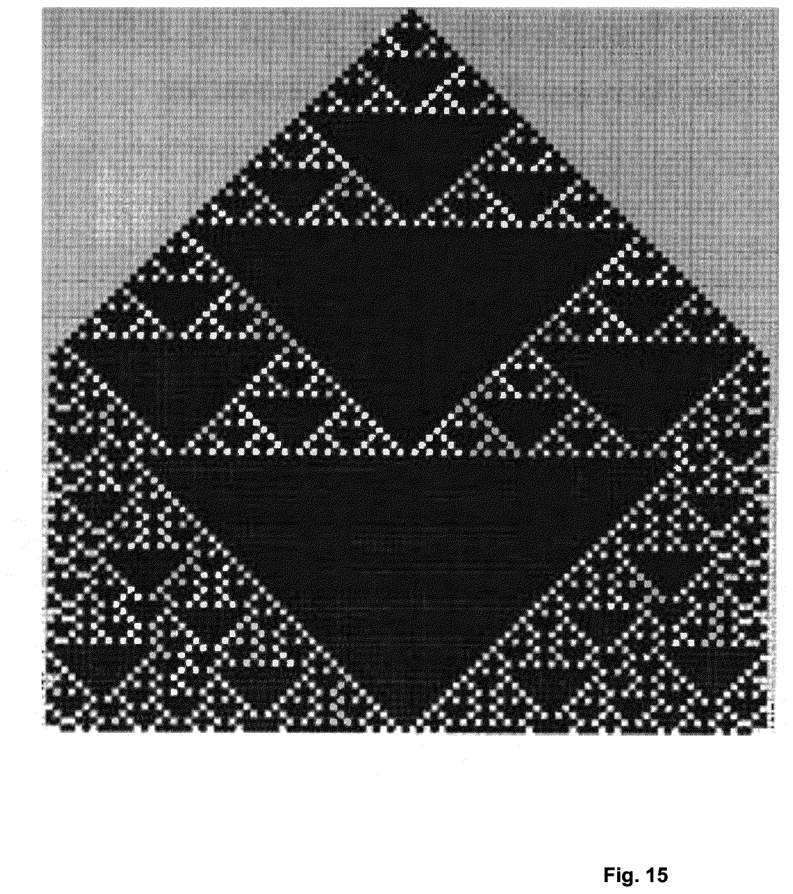

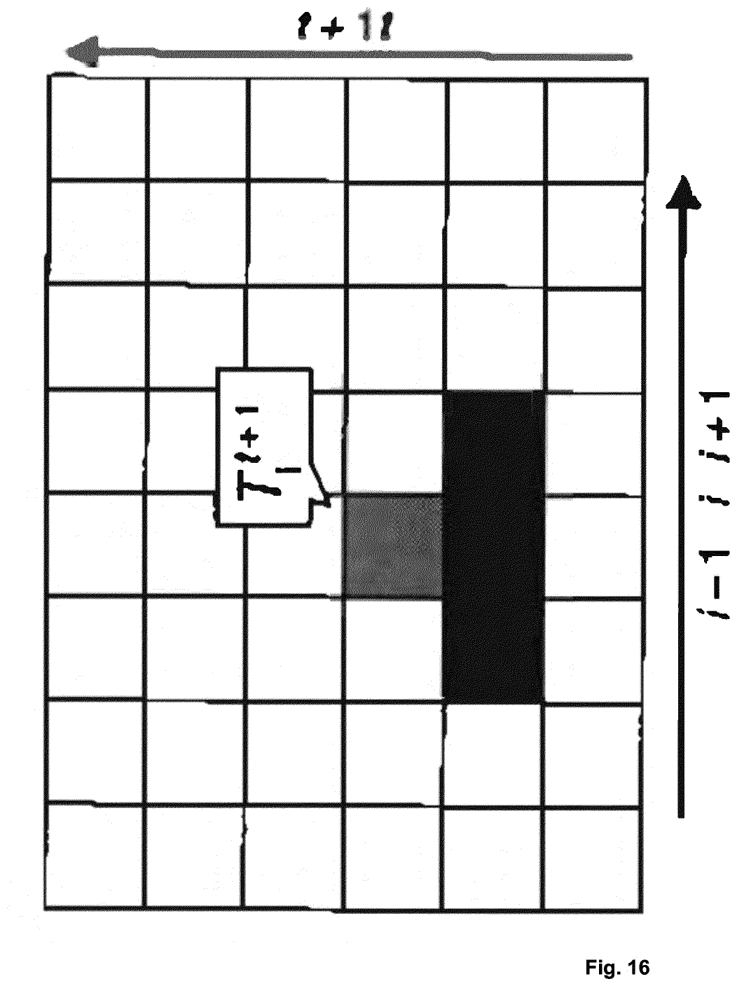

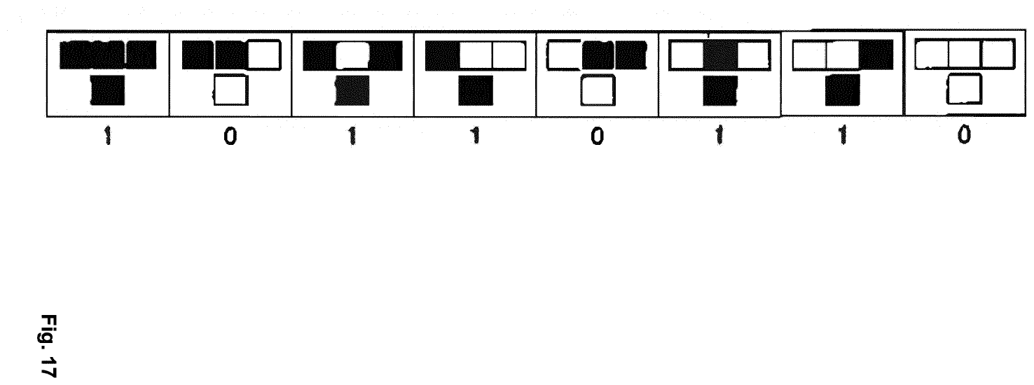

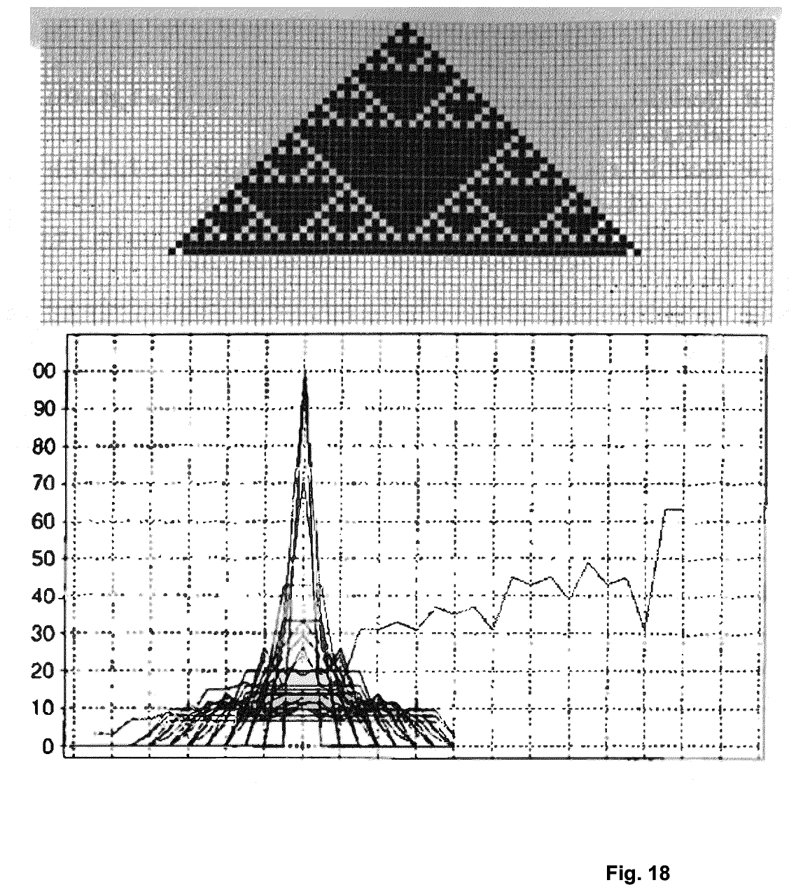

is the concentration, t is the time and is the diffusion coefficient. The translation of this relation into CA rules for dissolution simulation can be described using the finite differences method for generating the numerical processable solutions of the systems of differential equations. Using Wolfram's 1D cellular automata with rule 182 (10110110), as showed in FIG. 15, the transformation to the CA rules from the forward finite difference scheme can be presented as shown in FIG. 16. The target value for the differential relation is related to the sum of the function values Wolfram's ID cellular automata, which is also referred to as the forward 4-dot scheme for numerical solution of differential equations. Starting from the discussed CA cell development based on rule 182, the successive iteration (or value of the grey cell, see FIG. 16) can be generated from previous states of cells (black squares). Graphically the corresponding pseudo-code can be presented as shown in FIG. 17. The resulting numerically processable solutions of the diffusion relation through rule 182 might be presented in the form of a bell-shaped curve or cumulative curve vs. time. The resulting curves are shown in FIG. 18. As can be seen from FIG. 18, solutions with CA of classical physicochemical relations are possible and yield results very close to the analytical solution. As an advantage, such CA-based solutions have technically easy to handle boundary conditions, native to CA handling of localities, and respecting conservation laws. Because of the high flexibility of CA-based models of physicochemical phenomena, the CA-based algorithm is very promising and convenient as the basic model for formulation 13 design and modeling. The CA-based structure, according to the invention, is herein adapted for use as a simulation engine of the release profiles 12 from solid dosage forms.

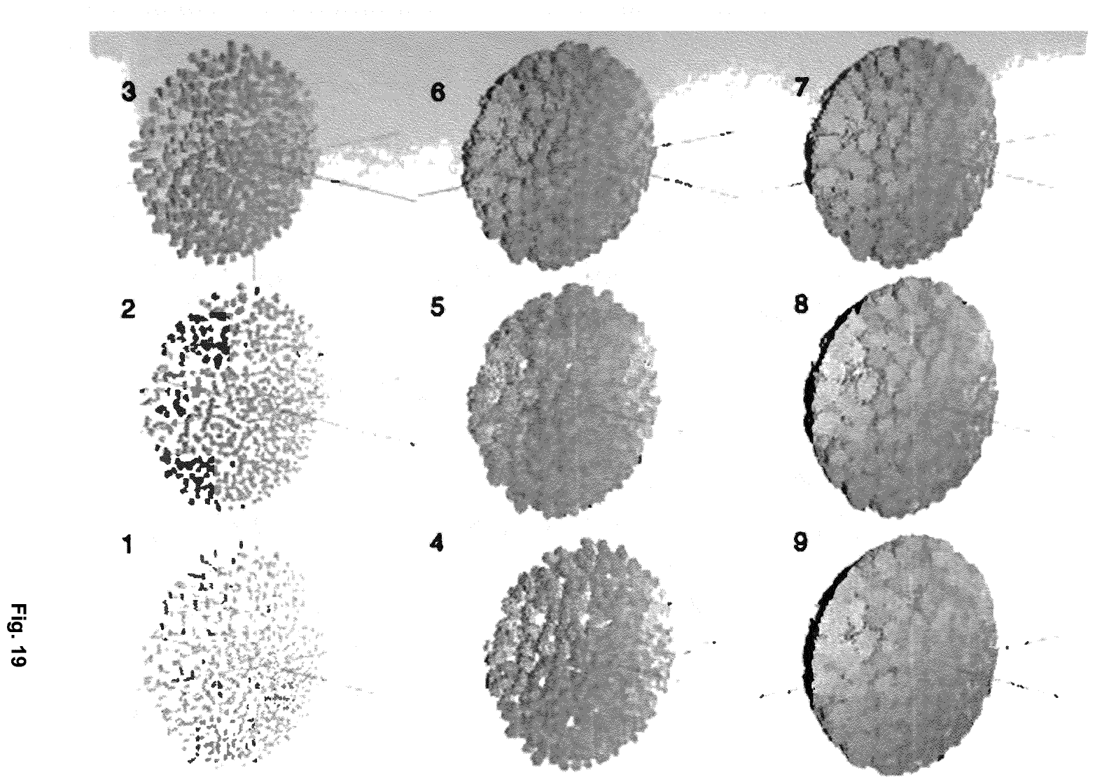

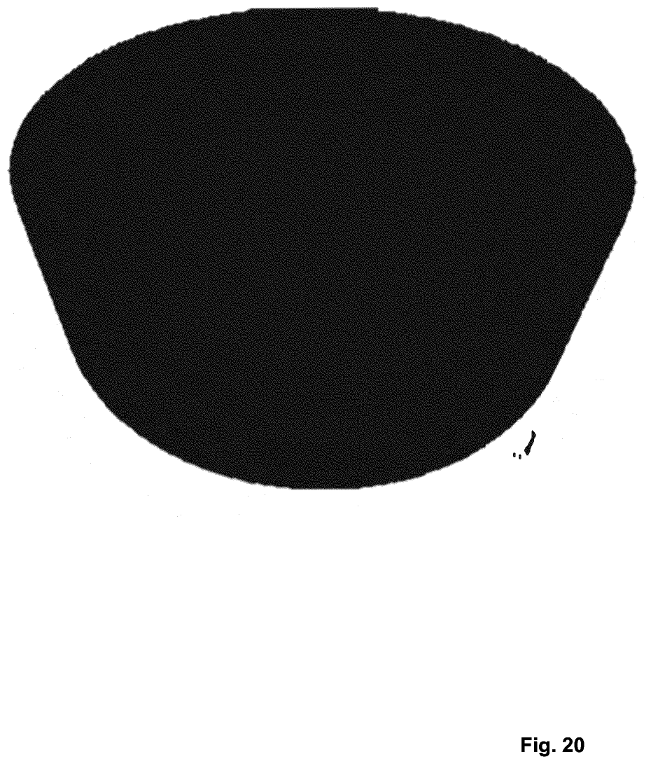

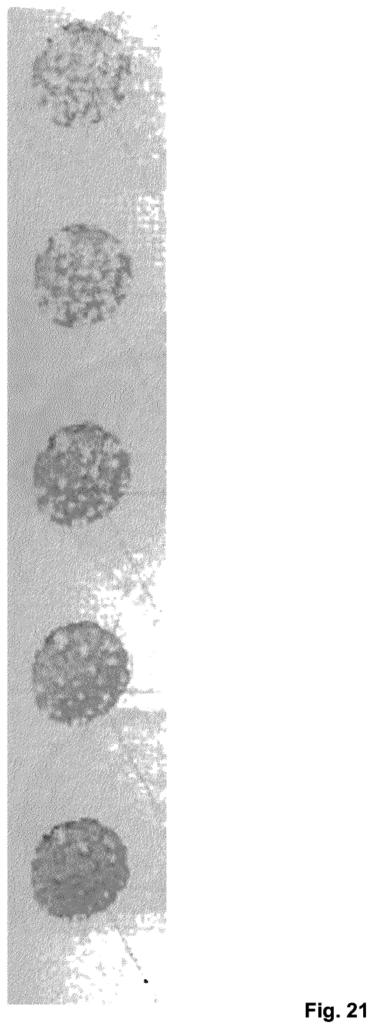

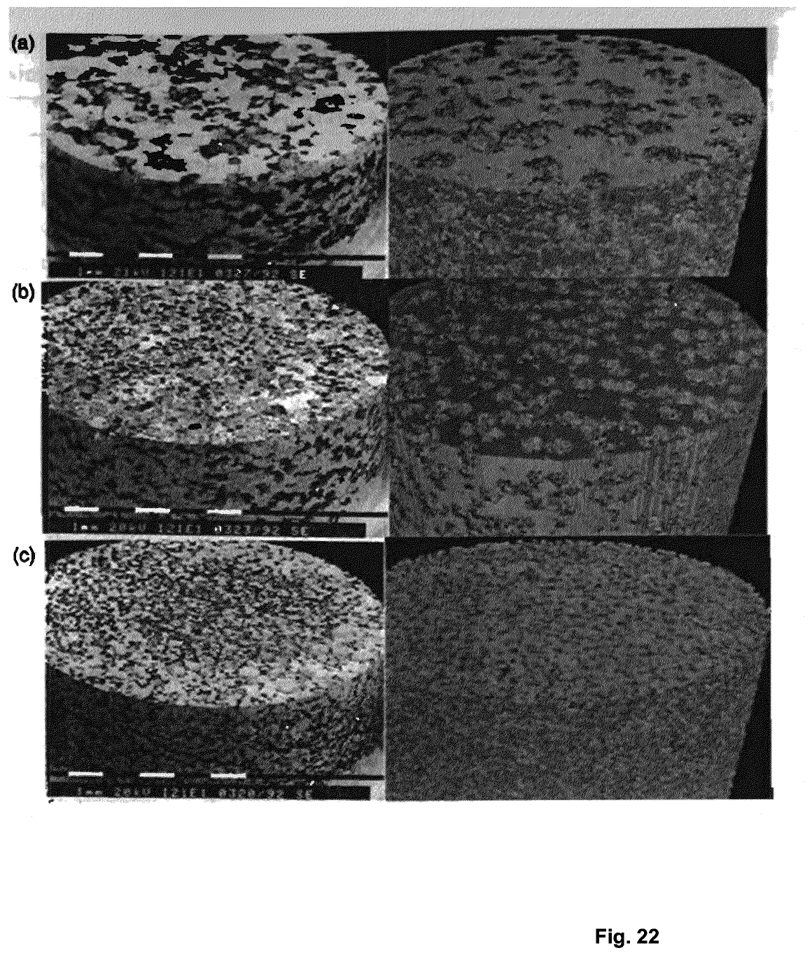

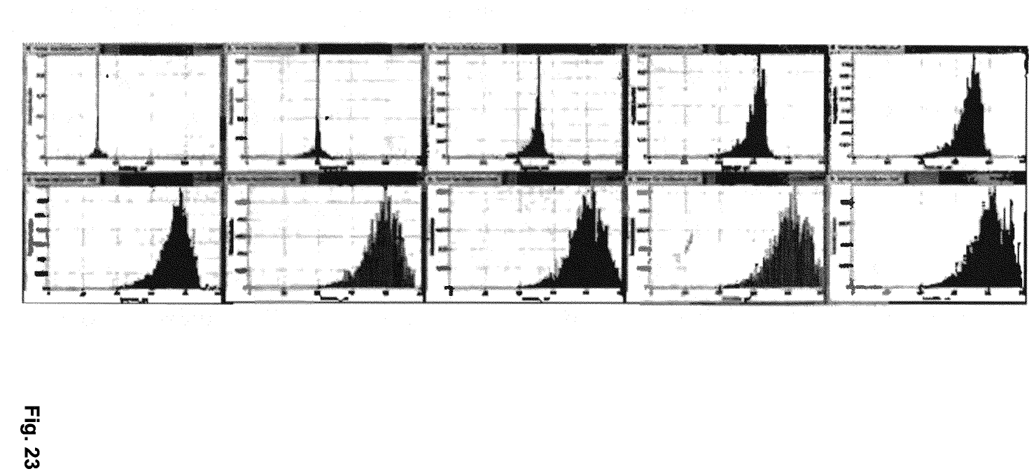

For cellular automata systems for tablet compaction, despite dissolution profile calculation being the main goal for simulations, the particle arrangement structure is another essential part of the whole system. Unlike natural compression, where the powder is first loaded into a die and then compacted due to the volumetric shrinkage, the particles are ‘grown’ to the target mass and size distribution within a final, desired, and rigid tablet volume. This processing is illustrated step by step in FIG. 19. The ‘growth’ process starts at ‘seeding’ the initial germ-particles of three individual cells in the centers of the future particles. The locations of the center particles are determined by an appropriately adapted sphere packing relation, as shown in FIG. 20. The initial centers will remain the barycenters of the developed particles. Spatial shift might occur because of constraints that force the particles to rearrange their locations and shapes. Once the centers are positioned, the subsequent ‘growth’ process is governed by implemented CA rules which transform the initial seeds into spherical objects. At this point growth is continued until the particle does not meet the boundary or another growing particle. At this event, diametrical growth is stopped, but the particle is trying to allocate the gained mass at the nearest empty place, i.e. the particle deforms. This technical approach allows the generation and simulation of a higher tablet density in the vicinity of contact surfaces, in a similar way as happens in reality during the compaction process. FIG. 21 shows the development of the solid structure rendered for visualization purposes at lower resolution, as a visualization of degradation of the porous network down co individual porous domains. The overall efficiency of such an inverted approach to compaction simulation is delivering very realistic and robust results when applied to hundreds of millions of cells as shown in the following example. The renderings from system 1 memory for tablets where the active ingredient was removed are shown in FIG. 22. On the left of each image, the real tablet with washed out API (caffeine) is shown, whereas on the right the corresponding result of a CA-based growth mechanism of the second task engine 17 respectively its particle arrangement and compaction generation and simulation is shown. The simulation was performed for three different size fractions of the API, which were visually compared with their real counterparts (FIG. 22). The particle size distribution of the simulated granules or particles varies during the growth steps. The resulting distribution function resembles the realistic figures (see FIG. 23). It could be expected that the size distribution development would be not very different to a delta function because of homogeneous time steps and equal rules application during the growth process; however, the heterogeneous and iteration-dependent constraints (walls and neighboring particles) change the distribution relation to Gaussian or similar type.

For creating a virtual tablet, the composition of the virtual tablet is set up together with the particle arrangement and compaction of the second task engine 17. This technical approach allows to include effects of unit operations on the formulation 13. However, the first step is to define the components of the test formulation. All that is needed for this realization of the system 1 is the true density value for each component, a user-defined color for visual representation, a name, and a type of the component. Depending on the type selected for a particular ingredient 111/112, different rule sets can be applied to the cell component. Different cell types allow the system 1 to predict and model a vast variety of different phenomena and behaviors of ingredients, retaining high flexibility and simplicity. The type depends on the properties of the substance. The selected types and their functional descriptions, which are used for the present embodiment variant of the system 1, can exemplary be chosen as “type #” to “description of compound properties” with 0 as pore, 1-9 as API, 10-19 as non-swelling, soluble fillers, 20-29 as non-swelling, soluble binders, 30-39 as non-swelling or negligibly swelling non-soluble fillers (MCC, etc.) absorbing water, 40-49 as hydrophilic swelling matrices (e.g. HPMC), 50-59 as spherical disintegrants, swelling e.g. starches, 70-79 as hydrophobic ingredients (glidants, etc.), and 200 as dissolution media.

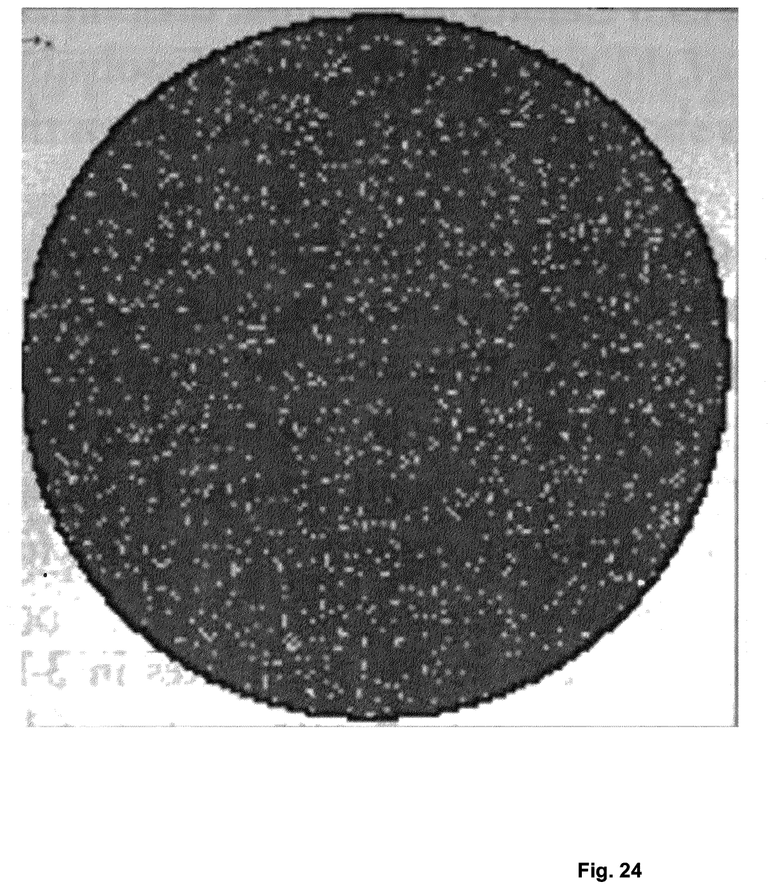

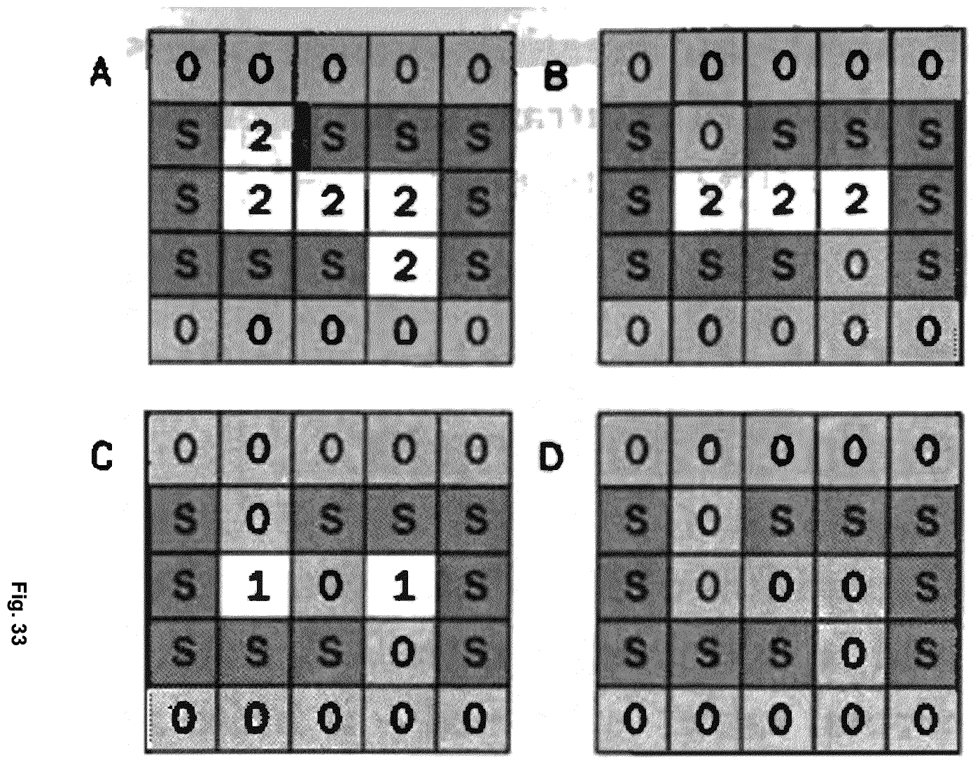

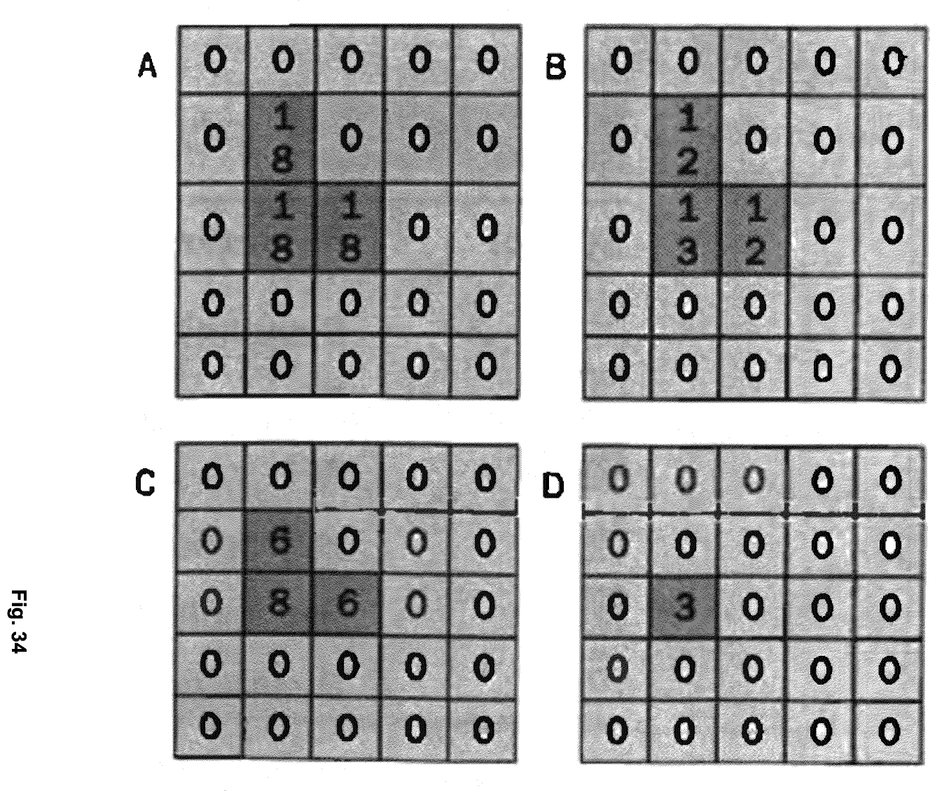

Each type is associated with its own rule set for the dissolution calculation with CA, as is shown and discussed by the FIGS. 33-38 below. As a second step (after components definition), the created tablet shape (0) is loaded into the second task engine 17 (PAC module). The discretized tablet shape in terms of CA-modeling and -generation is the constrained mesh where the CA will be defined. It is important to note that there are several possible ways, how the meshing and discretization of the smooth arbitrary geometry can be realized. The one which is used for the present embodiment variant is based on the ray-casting method. Filling of the components into the tablet in accordance with their individual concentrations and particle sizes finishes creation of the virtual tablet. This step allows very wide functionality, i.e. with this technical approach, it is possible introduce such effects as granulation or direct compaction, etc. Two main operations are defined while packing the components: seed distribution and growth as described above. The distribution function randomly distributes the compound for which it is applied. The number of cells to distribute is a function of concentration and true density of the component. At that moment, all components are defined in volumetric fractions. The system 1 distributes the number of cells needed to reach this mass. As an example, FIG. 24 shows the top view of a virtual tablet of pure caffeine with porosity of approximately 10% v/v encased in intrinsic dissolution constraints. The circular layer simulates an insoluble compound, which enables an intrinsic dissolution simulation by blocking the contact of the virtual liquid with dissolvable solid of the tablet. The same tablet is shown in FIG. 25, but this is the lateral view. If the distributed particles have a diameter bigger than unity, the ‘seed’ coordinates have to be arranged in such a way that imaginary spheres of the same diameter are densely packed. In this case, the spheres are also distributed randomly, but only in positions where spheres do not overlap with one another (see FIG. 20). Such a technical approach allows even distribution of the ‘nucleation’ or starter centers (the seeds) for further evolutionary growth of the particles or granules. Particle growing enables allocation of particles or granulates in 3-D tablet models. To achieve this, ‘seeds’ are placed into matrix cells positioned at the center of each particle. A seed is composed of three cells and is randomly oriented. Each particle in the matrix grows out of its own seed. The growing process is achieved by transforming each ‘empty’ cell in the Moore neighborhood of seed cells into a particle cell according to the target rule. In the same way, each newly created particle cell extends by transforming empty neighboring cells into particle cells. After a certain number of growing steps, particles will meet each other, i.e. they will share neighboring cells. In this case, particles will grow around occupied cells up to a certain extent. The resulting effect is a deformation aspect of particles, similar to real compacts. In order to respect the difference of hardness between components, the deformation potential of in silico particles should-be calibrated: The growing algorithm will stop when the volumetric ratio of each component is obtained and/or when the desired tablet porosity is reached. The result obtained with the particle growing algorithm is a discrete compacted mixture of ingredients ready for in silico dissolution in the cubic matrix.

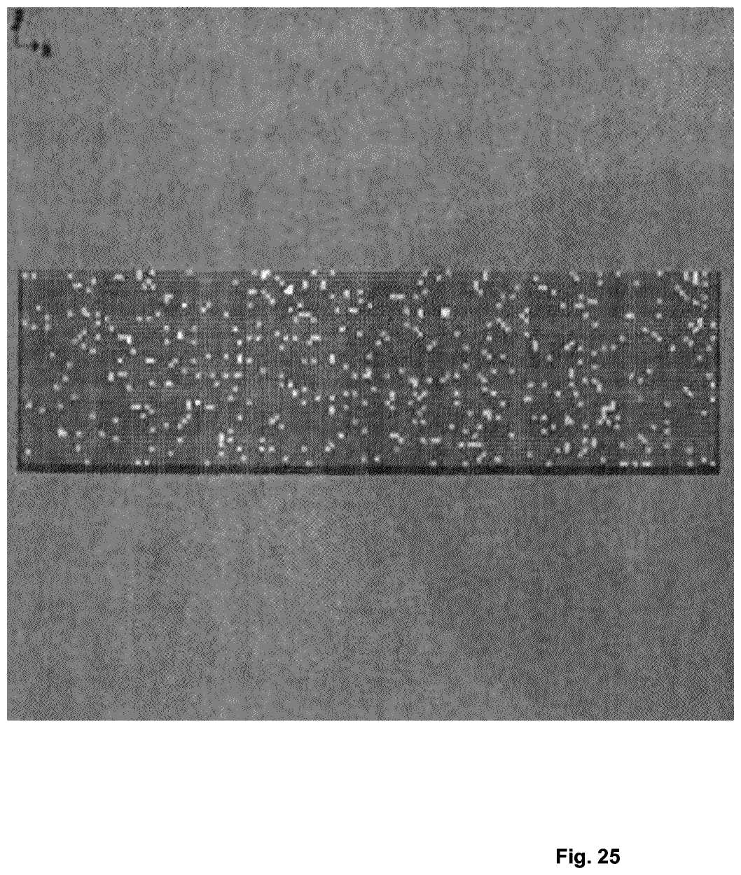

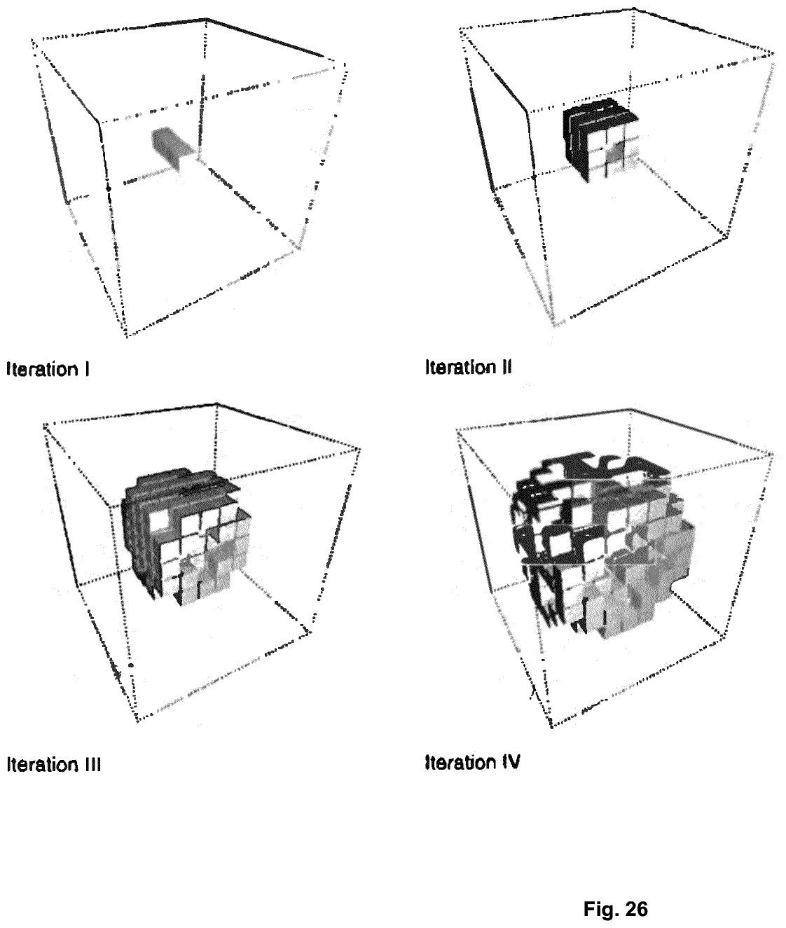

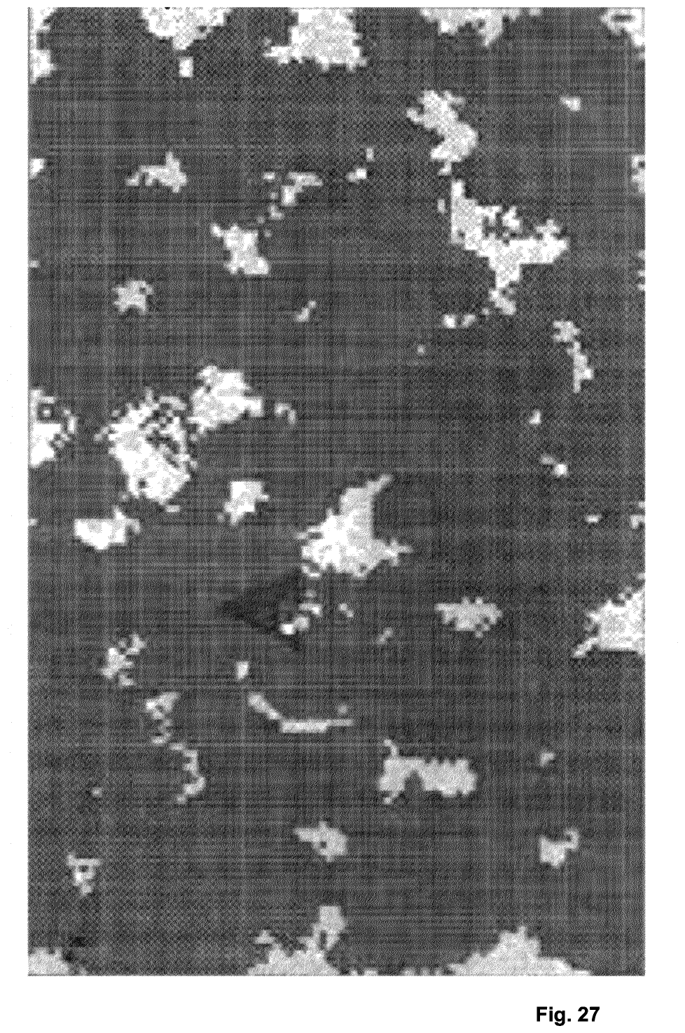

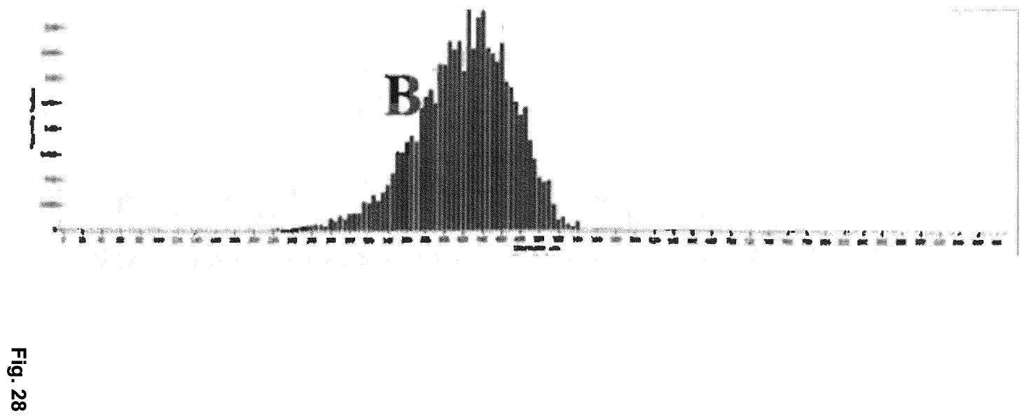

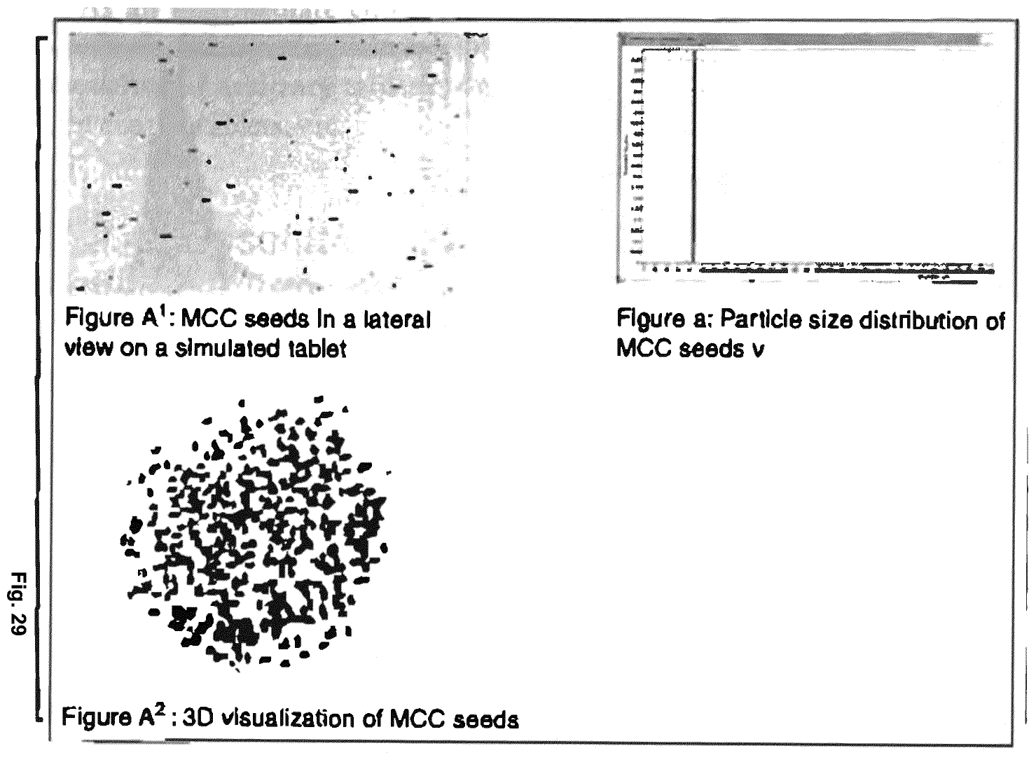

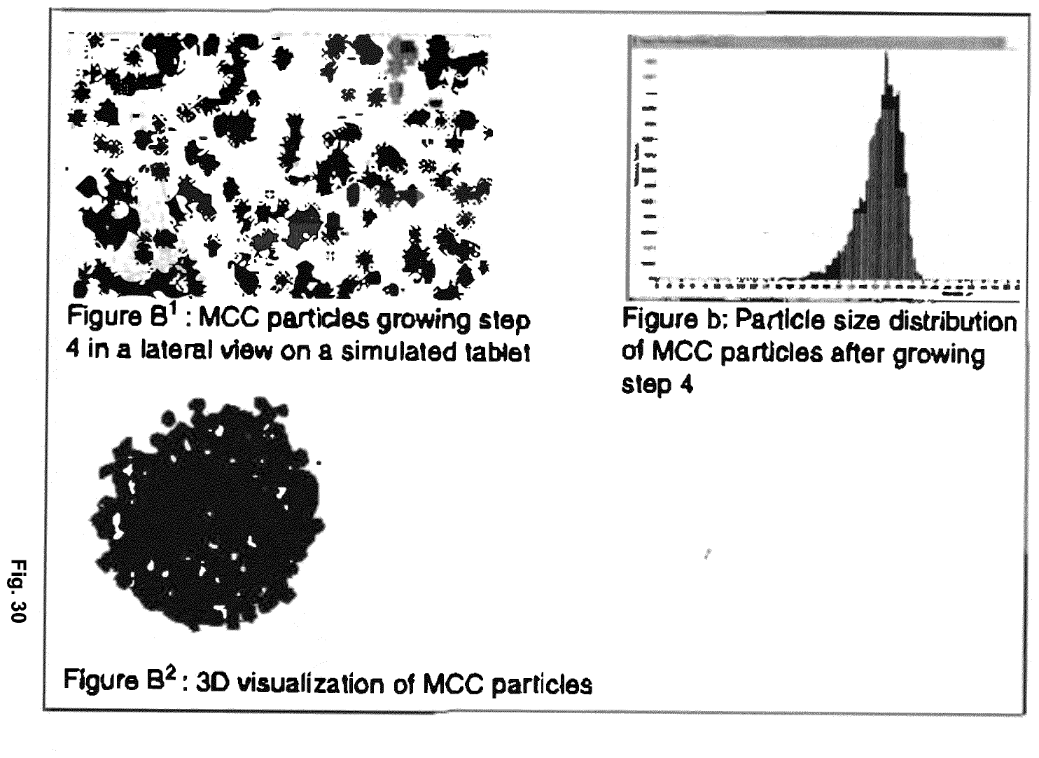

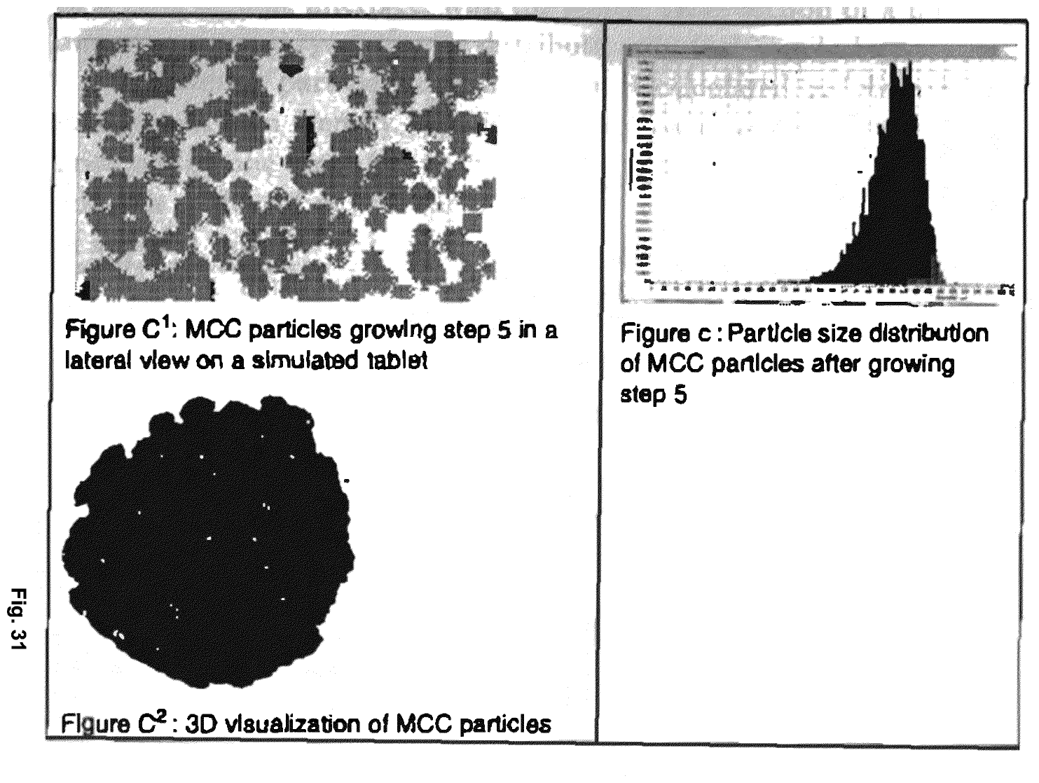

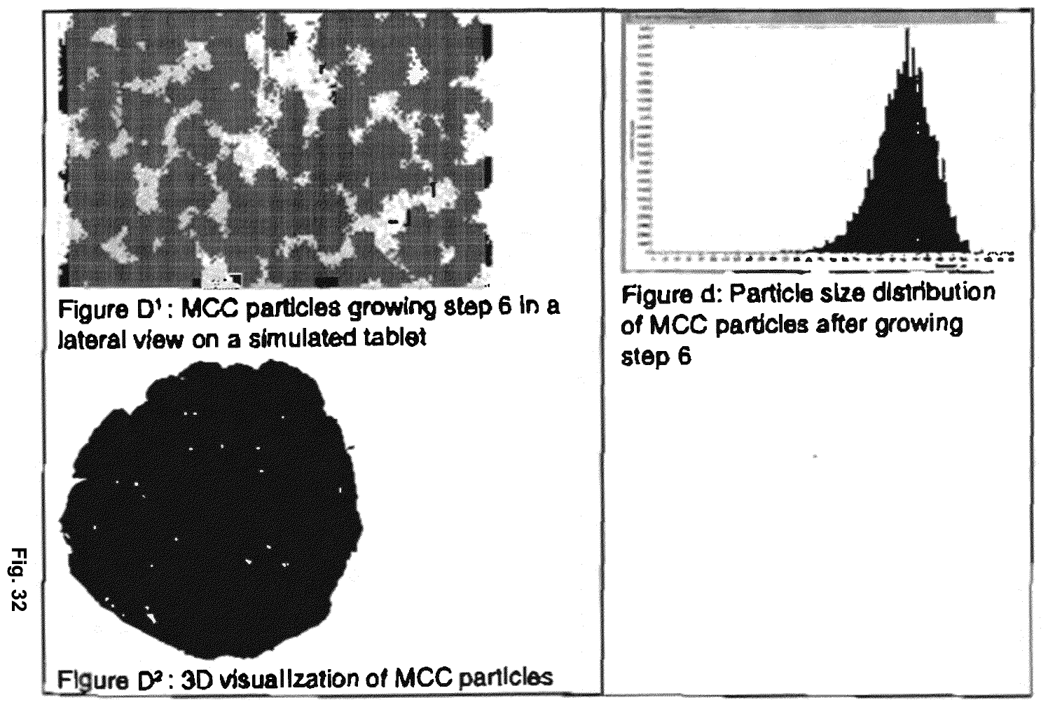

It is to be noted, that the growing process is based on cellular automata. To illustrate the growth of one seed, FIG. 26 shows the growth of a seed with an arbitrary-selected rule set. This rule set is growing a sphere-like particle out of the starting seed in four iterations. The system's 1 hardware and/or software based realization of the growing procedure can be similar to the one in FIG. 26, but with a different rule set. As visualization, a screenshot of a PAC module 17 interface, with the lateral cross-section of a tablet (FIG. 27) and the generated particle size distribution (FIG. 28). The resulting particle size distribution can be generated as follows: every seed has its own identity. This identity is passed on to the cubes which arise out of one seed during the growing process (the ‘offspring’). The volumes of the cubes with the same identity are added and the diameter of a sphere with this volume is then taken into an account for particle size distribution. This is process is done for all particles. FIGS. 29-32 shows the growing of micro-crystalline cellulose (MCC)-simulating seeds in a virtual tablet (figures A¹-D¹ and A²-D² of FIG. 29-32) and the associated particle size distributions (figures a-d of FIGS. 29-32). FIG. 29 shows only one peak in the particle size distribution plot, because all seeds are composed of three unit cubes which makes them all the same size. Figures A²-D² are graphic visualizations of virtual tablets which are similar to the tablets shown in figures A¹-D¹. The evolution of the particle size distribution from a single delta function (the starting peak) into a bell-shaped curve could be explained by distribution of possibilities to grow for all particles. This is indeed a stochastic process and it can be observed an interesting transition from an ordered system at initial conditions into a fully random and chaotic arrangement. As the present realization of system 1 shows, the applied and developed CA-based methods for creating virtual tablets are extremely flexible and allow modeling of arbitrary tablet geometries, including multiple layer tablets, dry coated tablets, etc.

For the dissolution profile generation by means of the present system 1, i.e. the cellular automata, the following has to be considered. The generation of the release profile 12 from a virtual tablet is a separate task and can e.g. be fully based on the CA rules. The calculation matrix, where the virtual tablet is defined, is finite and cubic, as well as the grid which forms the smallest units of the matrix: the unit cubes. Time is also discrete in CA, wherein one time unit corresponds to one update of the whole system 1 which leads to the next generation matrix. The unit cubes have different types with different properties, which are represented in rule sets. The types, available for this embodiment variant, have been exemplary listed and discussed above, and correspond to the behavior of different components in a real formulation 13. For a better understanding, the meaning of cell types and their corresponding rules are described in the following. The used cell types can e.g. be generalized for better understanding of the typical rule sets used to generate the release profiles 12, which are showing the possibility and the performance of the system, in assigning the following symbols to the representing cell types: 0 to dissolution medium, 2 to pore, 5 to API, 6 to hydrophobic offspring cell, 3 to swellable compound cell, and D disintegrating compound cell. These symbols and number-coding can e.g. further be used in visualization of the figures in FIGS. 33-38.

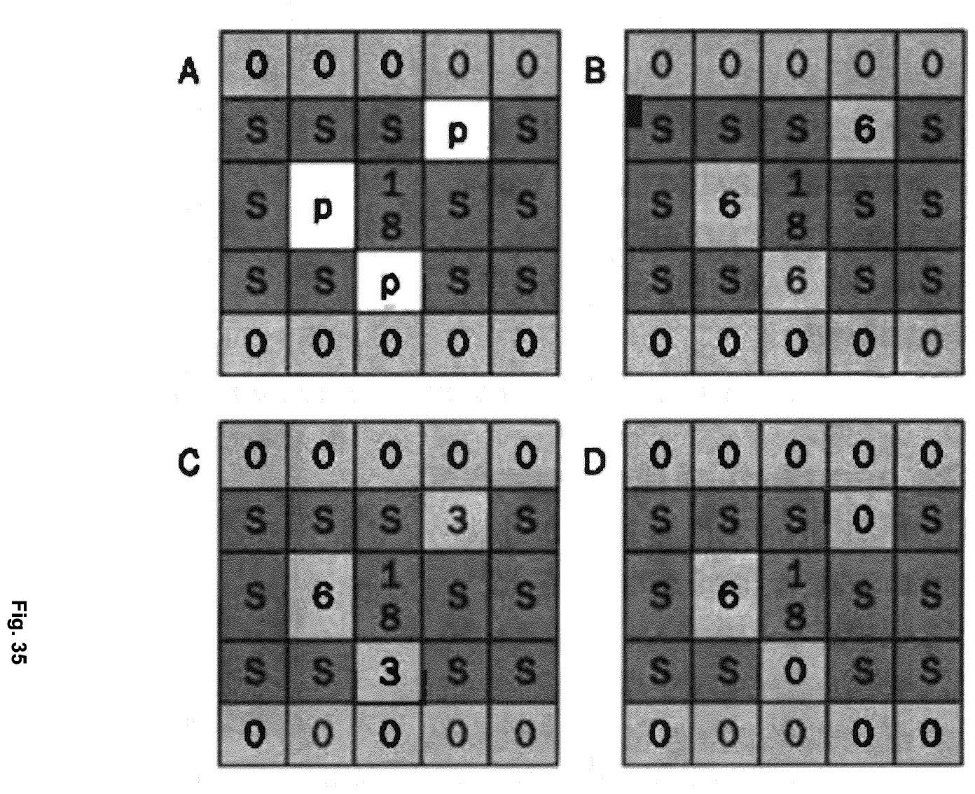

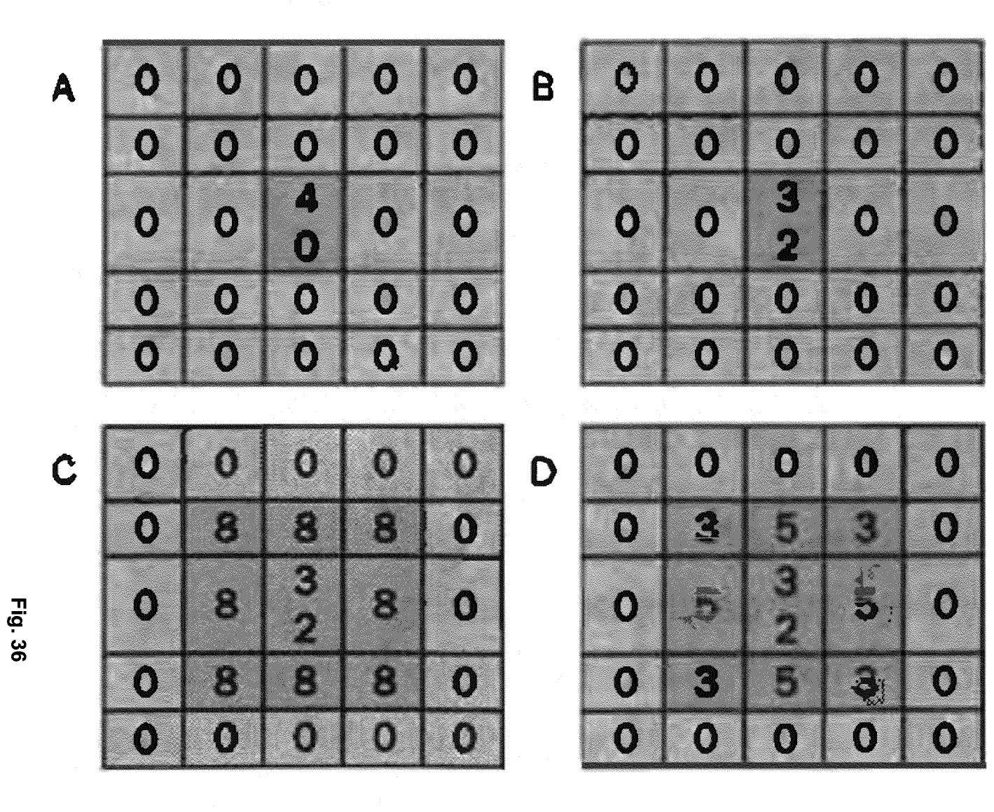

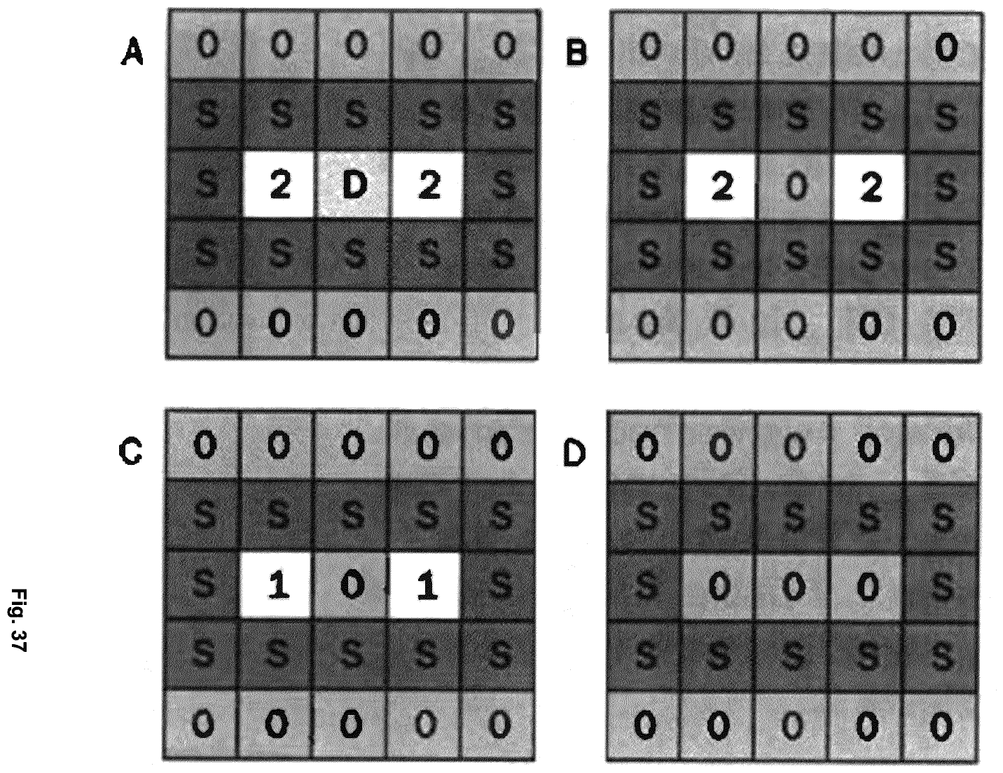

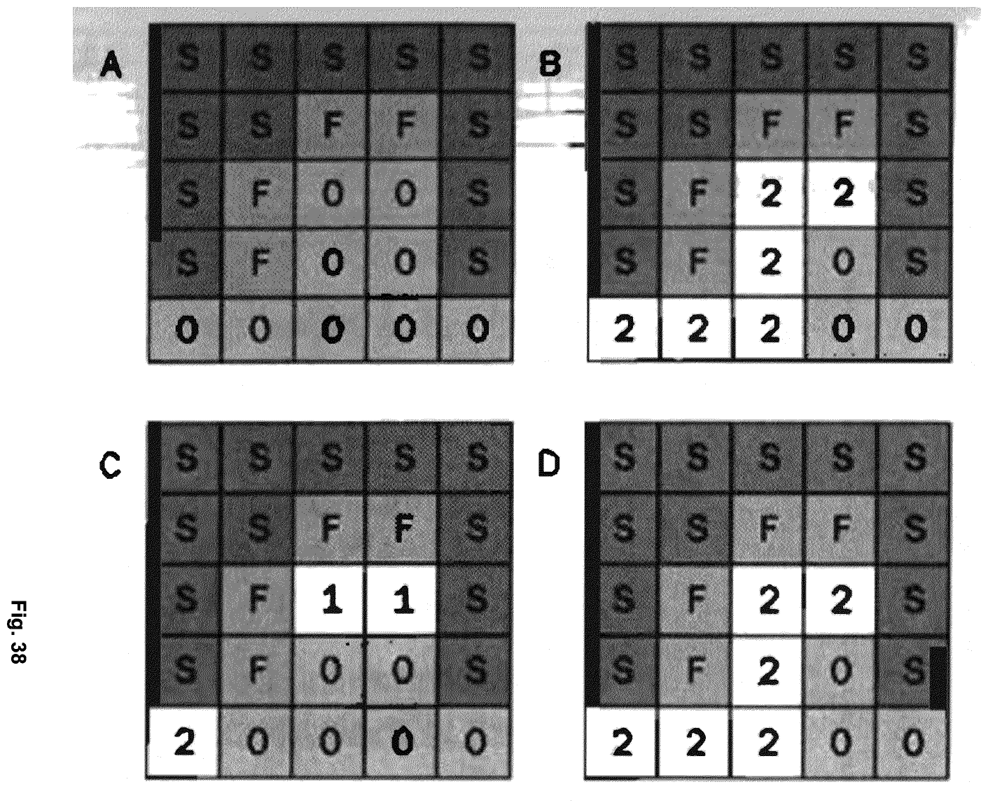

FIGS. 33-38 show the basic CA-update rules for different types of the components, i.e. (i) pores, (ii) Active Pharmaceutical Ingredient (API), (iii) hydrophobic API (hAPI), (iv) Swelling compound (Sw), (v) Disintegrants (Dis), and (vi) Fillers absorbing water (e.g. MCC). For (i), the pores, the property of pore cells is regulated with C1ₚₒᵣₑ. To mimic the pores being wetted with a certain speed, pore cells are given a value (C1) which defines how many generation steps are necessary to change a pore cell with contact to a liquid cell in the Moore neighborhood into a liquid cell. The higher the value of C1ₚₒᵣₑ, the more iteration will be needed to change the type of a pore cell. In other words, the more hydrophobic the substances comprising a tablet formulation 13 are, the slower will be the wetting process and the higher C1ₚₒᵣₑ needs to be for an accurate simulation. In this case, the primary constant C1 is the measure for hydrophobicity of the API or other ingredients. As illustrated in figures A-D of FIG. 33, every liquid cell in the Moore neighborhood of a pore cell subtracts one unit from C1ₚₒᵣₑ value until C1ₚₒᵣₑ=0 and the cell changes its state to that of a liquid cell. For (ii), the Active Pharmaceutical Ingredient (API), the solubilization property of a non-hydrophobic API cell is characterized with C1_{API}, no C2 is necessary. C1_{API} defines the number of iterations necessary to change the solid state of the cell to a liquid state. As for the pore cells, every liquid cell in the Moore neighborhood subtracts one unit from C1_{API} at every iteration. As soon as an API cell changes its state from solid to liquid, one unit is added to the dissolution counter, which corresponds to the- amount of drug dissolved in the experimental dissolution test during the same time interval. In the example on the right-hand side, an API particle getting dissolved is shown. Two units would be added to the dissolution counter before step D. For (iii), the hydrophobic API (hAPI), the difference to API cells is that a hAPI cell is given a C2_{hAPI}. Hydrophobicity is imitated by giving the pore cells in the Moore neighborhood of an API cell a new C1ₚₒᵣₑ. C1ₚₒᵣₑ is generated by dividing C1 hAPI by C2_{hAPI}. In the example of FIG. 35 on the right-hand side, a hAPI cell with C1_{hAPI}=8 is surrounded by pore cells (p) and solid cells (S). C2_{hAPI} is 3. As a consequence, pore cells in the Moore neighborhood receive a C1ₚₒᵣₑ of 6. This step is not the end of the first iteration, but step 1.2 (see the generation cycle of the system's 1 dissolution generation, as given below). The end of generation one is shown in figure C. For (iv), the Swelling compound (Sw), this cell type is used to simulate compounds getting more voluminous when coming into contact with water. Liquid cells in the Moore neighborhood are given (similar to hAPI cell type) a C1, which is calculated by dividing C1_{Sw} by C2_{Sw}. The smaller C2_{Sw} the higher is the expansion capacity of Sw cells. In figures A-D of FIG. 36, 2 generations are shown, whereas the initial C1_{Sw}=40, C2_{Sw}=3. Figure A shows the initial state, Figure B shows the decrement of C1_{Sw} (step 2 of the generation cycle of the system's 1 dissolution generation, as given below), and figure C shows the matrix after step 3. Figure D shows the matrix after step 2 of the second iteration. This procedure enables simulation of complex swelling behavior with particles growing to a certain extent and then remaining In equilibrium between swelling and getting dissolved. For (v), the Disintegrants (Dis), the approach of disintegration simulation, as illustrated here, is an attempt to simulate a quick exposure of API cells to liquid as is the case for disintegrating formulations. The constants of Dis cells are both zero. This results in an immediate change of state after the first iteration as seen in figures A and B of FIG. 37. This change happens no matter whether or not the Dis cell has contact with any other liquid cell. In the illustration pore cells are given a C1ₚₒᵣₑ of 2, the solid cells are not given any attention. This simplified disintegration rule set has limited usage and has to be expanded by movement of the cells during the simulation. For (vi), the Fillers absorbing water (e.g. MCC), in the case of sorption fillers or similar formulation components, the liquid in the neighborhood will be converted into pore until the sorbent cell is completely saturated (regulated by the C2 value). Once the saturation event is triggered, the filler is no longer changing the state of the liquid cells, hence allowing the liquid cell to dissolve adjacent API or other component cells. Figure B of FIG. 38 shows the conversion of liquid cells into pore cells. During the same iteration, the value decrement is done (Figure C). If the sorbent cell F is not yet saturated, liquid cells in the Moore neighborhood will again be changed into pore cells (Figure D).

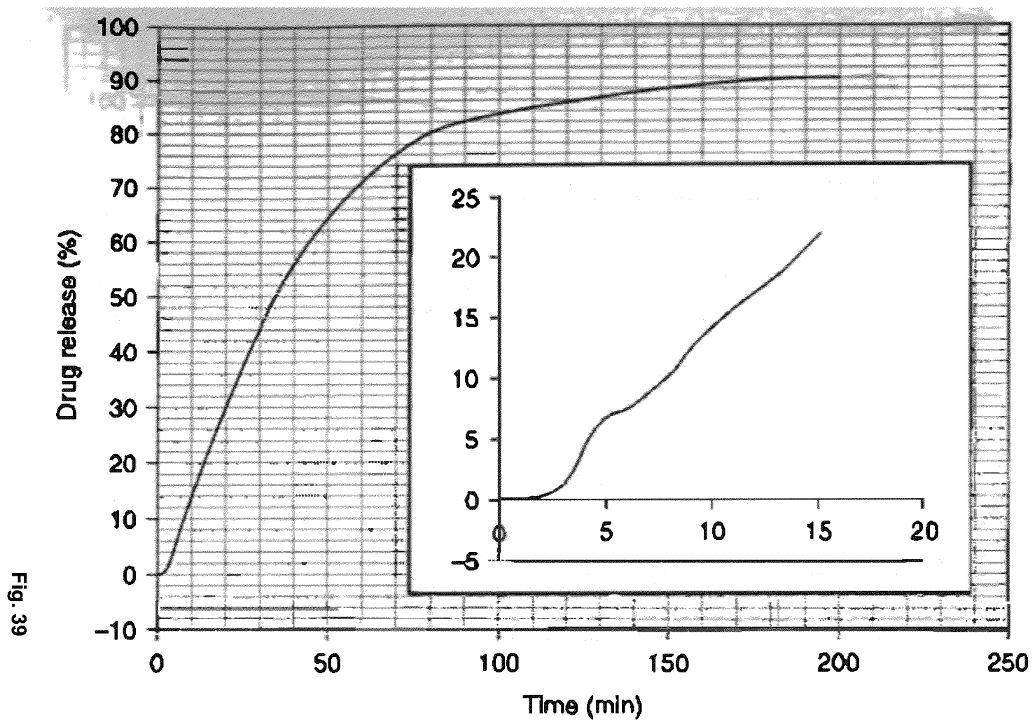

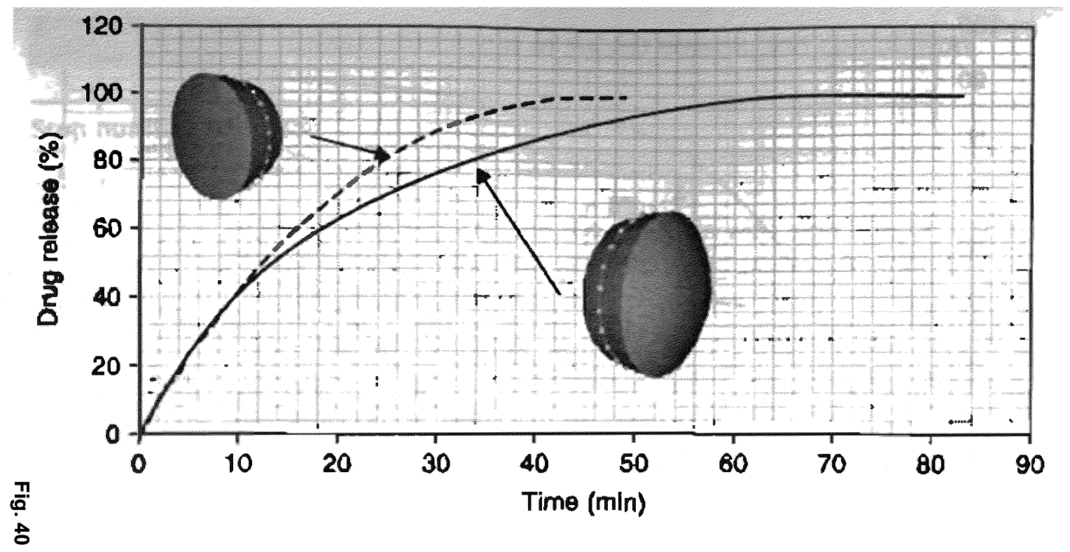

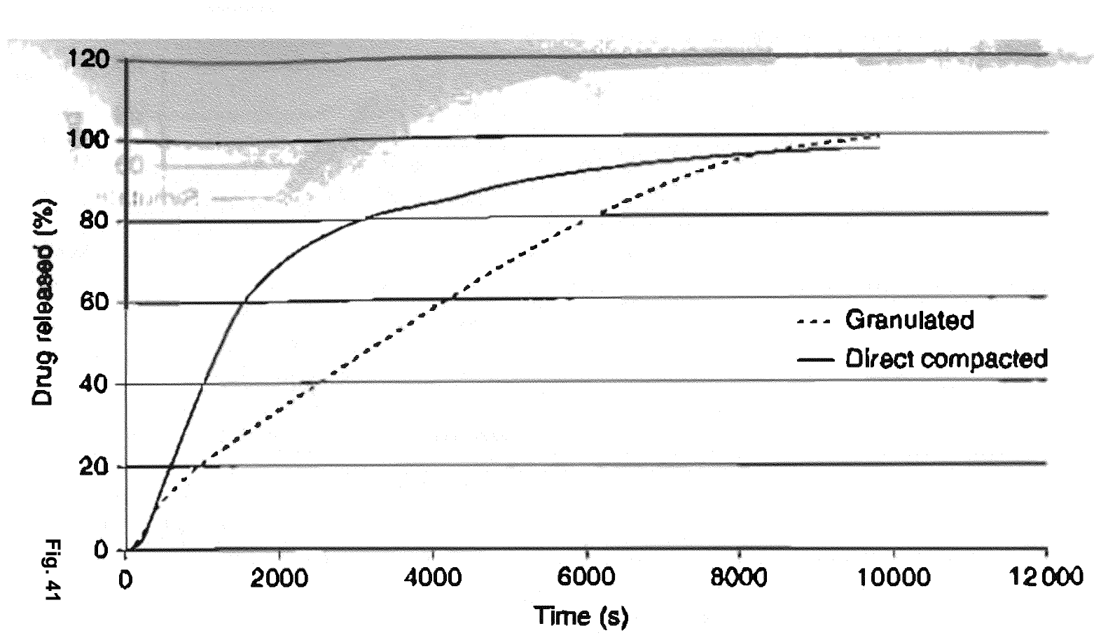

As can be seen from FIGS. 33-38, the rule sets are dependent on the primary and secondary constants, introduced into these CA-based dissolution models. Technically speaking, every cell contains two constants: the Primary Constant (C1) and the Secondary Constant (C2). The definition of the constant is as follows: C1 and C2 are constant for every unique substance for time=0. For any successive rime step those values are subject to change during the generation according to the rules as in FIGS. 33-39. Each cell type and subtype has its own constants, which need to be calibrated prior to tablet dissolution simulation. The calibration is best done by comparing experimental dissolution profiles co in silica generated dissolution profiles for a tablet with equal shape and porosity. The constants C1 and C2 are adapted until the simulated profile best firs the experimental one. C1 indicates the number, of iterations needed to change the state of a cell from solid co liquid. The lower the value for C1, the fewer iterations are required. The physical meaning of C1 is reciprocal solubility per unit of time. C2 does not influence the state of a solid cell itself, but has several different functions which depend on the cell type and are explained and illustrated in the FIGS. 33-38. The generation and calculation cycle, i.e. the sequence in which the rules are applied on the system, is the same for all dissolution simulations and is structured as step number assigned to action with 1.0 assigned to initial updates; 1.1 to type 30 value increases, i.e. all cells with type 30-39 (e.g. MCC) will give a defined value to liquid cells In their Moore neighborhood; 1.2 to pore hydrophobization, i.e. pore cells neighboring a hydrophobic API cell are given a defined value to simulated hydrophobicity of API; 2 to value decrements, i.e. liquid cells subtract one unit from any solid cell in their Moore neighborhood; 3 to swelling propagation, e.g. the behavior of a swelling compound; and 4 to final update of the matrix, i.e. all cells with a C1 of zero are changed into a liquid cell with the associated properties. Also, the number of API cells which changed their state due to virtual solubilization is added to the ‘dissolution’ counter. The release profile 12 of the API is generated for each time point in accordance with an initial set of conditions. The rules for CA matrix, described in FIGS. 33-38 are applied at each iteration seep, and the statistics of CA cell count are collected. The amount of drug released is calculated in accordance with the number of cells which have changed their state from solid to liquid. The release kinetics can be significantly altered by the particles located right on the surface, i.e. in direct contact with dissolution media. This effect is taken into account by the CA model provided by the system 1 and is illustrated in FIG. 39. The enlargement section of the simulated release profile 12 shows the release of the drug which is located directly on the surface of the compact. Usually, these effects are overlooked for immediate release formulations due to sampling times resolution; however, extended release formulations with hydrophilic matrixes have often displayed an initial ‘burst’ effect which is governed by a fast penetration of a dissolution medium into the surface layer of a compact. Another important property of a pharmaceutical compact is its shape 13111/13121/1324. The shape 13111/13121/1324 adjustments are often required not only because of marketing issues, but also because of technical requirements as coating 1323 necessity, etc. However, a formulation 13 might be sensitive to the shape 13111/13121/1324 alterations; hence changes in the release profiles 12 are expected. The CA-based model generations of drug release emulation are capable of displaying the shape 13111/13121/1324 effect on the release profile 12. The generated release profiles 12 for identical formulations 13 and identical compact volumes and porosities are shown in FIG. 40. This property of the CA-based release model generations is very helpful for research work on geometrically controlled drug delivery devices and formulations 13. It is known that different unit operations such as direct compaction, or wet granulation, result in differences in particle sizes and different particle arrangements, which need to be taken into account while doing simulations, generation and modeling. Direct compaction assumes intermixed particles of API and excipient to form a compact, the wet- or dry-granulation process clearly specifies the inner and outer phases. In order to simulate granular arrangement of particles and corresponding release profile 12 changes, the ‘Swiss cheese’ procedure could be used. Following its name, the ‘Swiss cheese’ structure is developed by growing only granules until a specified or desired size distribution. Once the granules are created, the remaining space is filled completely with auxiliary (starting) material (keeping its initial size) and the larger-sized granular material is removed. The remaining structure very much resembles Swiss cheese, hence its name. The holes in the ‘cheese’ are filled with a composition of the inner phase and the auxiliary material is exchanged with a composition of the outer phase of the tablet formulation. FIG. 41 shows the drug release profiles 12 of a relatively well soluble drug substance in two identical compositions, where the only difference in the formulations 13 is of the unit operations involved: one simulates direct compaction and the other is granulated. This example shows chat release curves could show significant differences depending on the unit operation employed, and gives a good approximation for investigation of the unit operation influence on the final release profile 12 of the drug. As it can be seen from system's cell types, as described above, and basic CA-update rules for different types of the components, as also described above, the number of basic CA types used for modeling the dissolution behavior of tablet formulation is not extensive. However, those types and the rules with those types calculated during the CA-update cycle allows modeling of a very large number of phenomena which occur during dissolution of a tablet formulation.

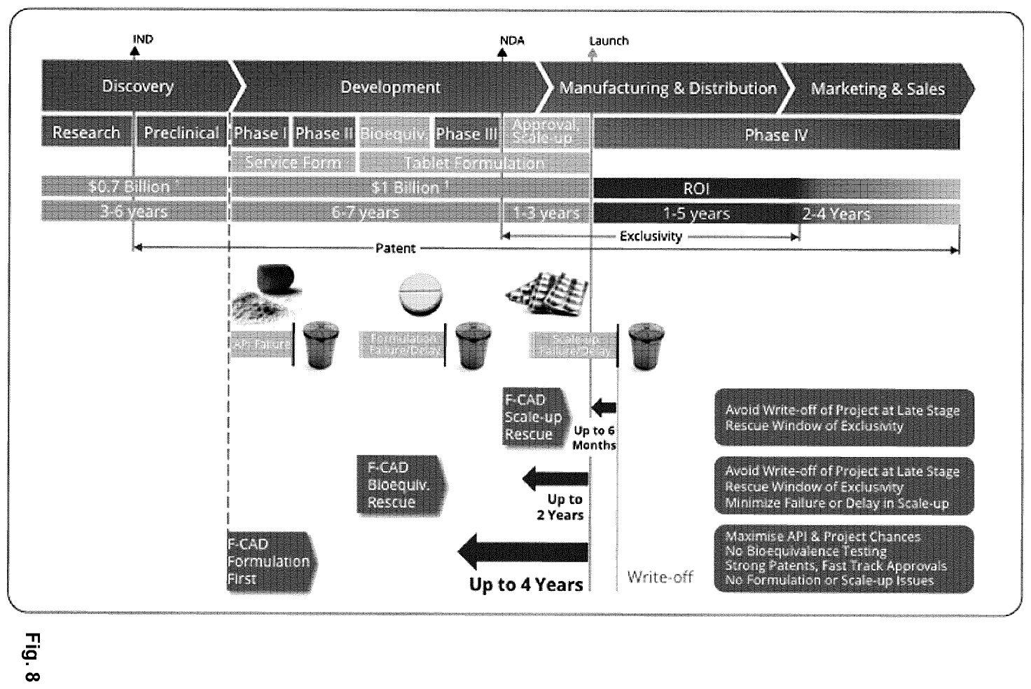

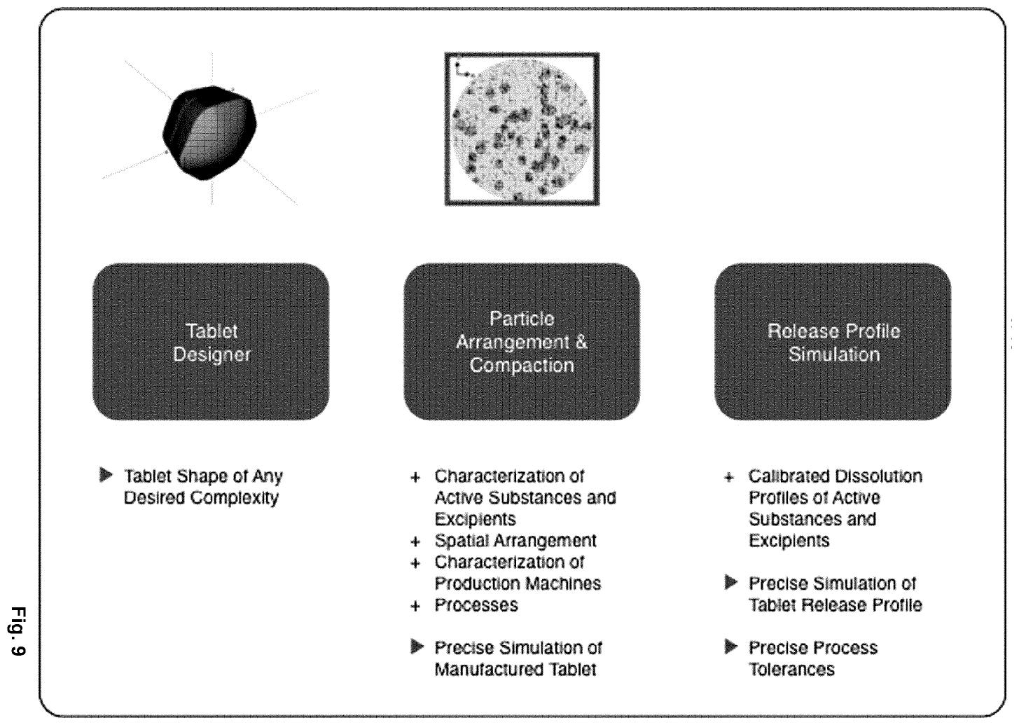

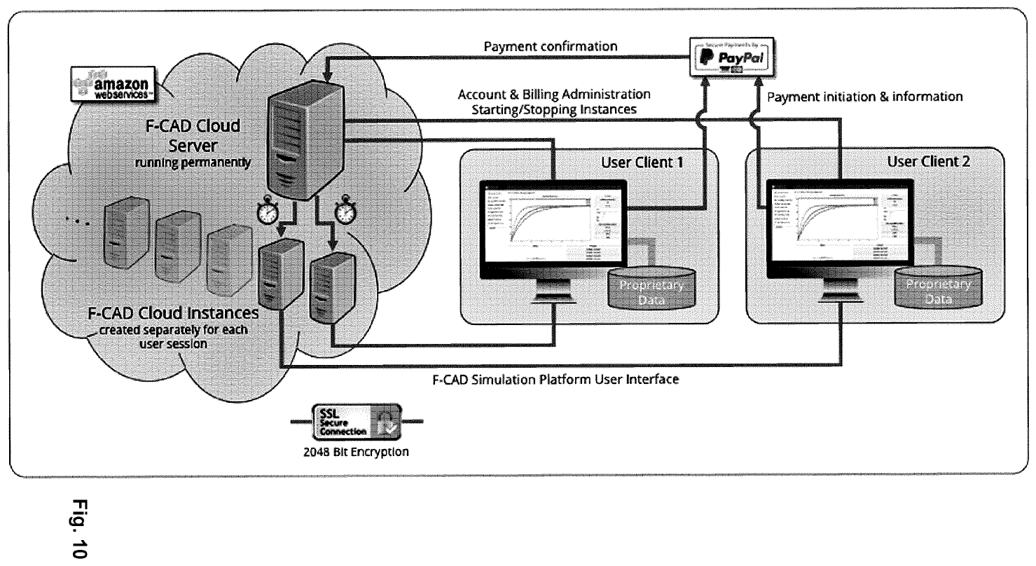

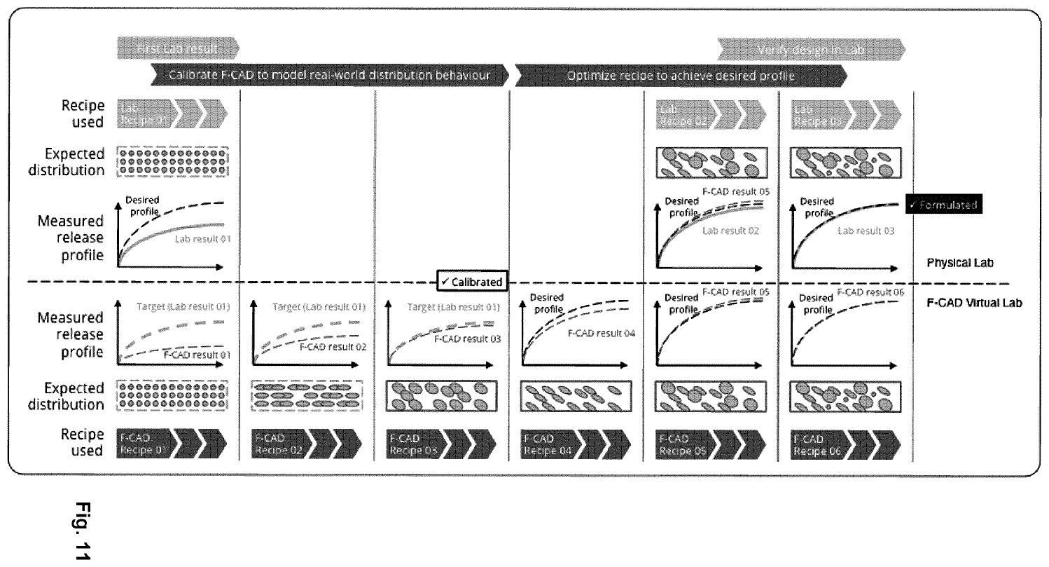

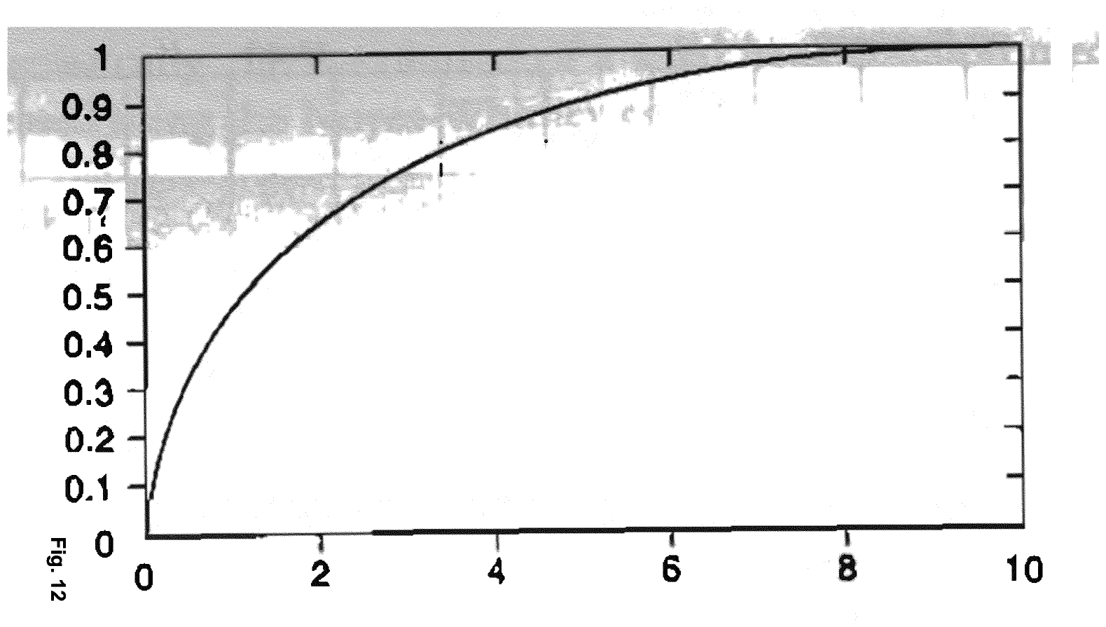

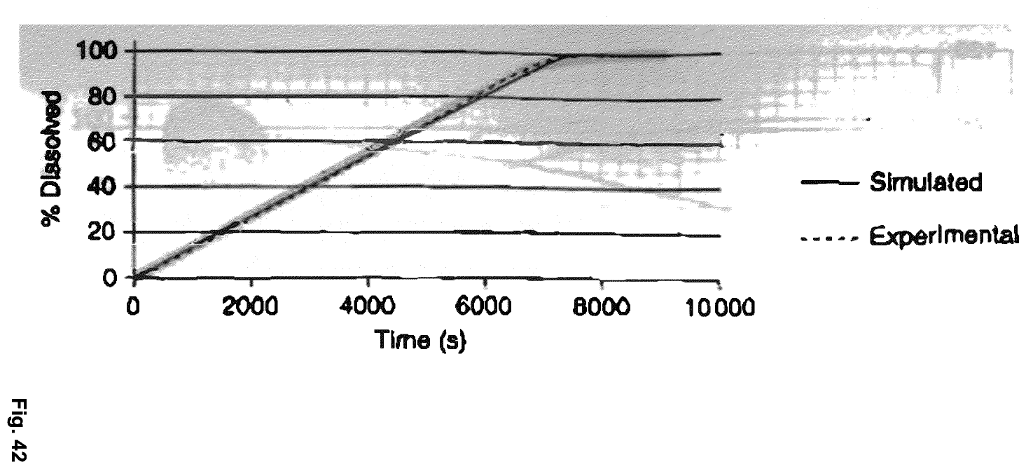

To verify and calibrate the prediction and modeling of solid dosage forms with the cellular automata based system 1, several laboratory experiments with model drug (caffeine and proquazone) formulations 13 were carried out. As an initial step the intrinsic dissolution rare (IDR) profiles were acquired. The usual purpose of chose experiments, where pure API is compacted into a tablet with constrained surface, is to measure the dissolution kinetics of the API in the target buffer and to obtain the calibration value of C1. The value for C1 is obtained either by manual fitting until the experimental and simulated curves match or by applying optimization algorithms such as the Nelder-Mead simplex-based method. The machine-assisted C1 search may be preferable in cases where the formulation for IDR-profile determination is necessary, e.g. low compressibility or compatibility of the target API. Simplex-based methods are capable of finding two or more optimal parameters, i.e. in this case the C1 for both API and the excipients could be found. As a disadvantage of this latter technical approach, the ‘entrapment’ of simplex in local extremum a should be noted. FIG. 42 illustrates the two IDR-profiles: one is the average of the caffeine release profile (including the error bars) and the other one is the simulated curve of the same formulation with the same geometry constraints {see FIG. 25). The two curves are practically indistinguishable from each other, which is shown as complete overlap of the curves. It has to be kept in mind that the CA-based generation methods are not based on the fitting approach; therefore, to obtain the simulated release curve the time seep has to be defined. In the presented curves, the time step was set to is, i.e. each update cycle of the whole calculation matrix corresponds to 1 s of real dissolution time. In such a case, it is very advantageous to use massively parallel processing realizations of the CA-based dissolution generations, as e.g. software-based algorithm processing. As a performance example, the parallel version of CA-based proquazone IDR-profile (FIG. 7.25) could be generated for 300,000 s (approx. 3.5 days) in only 10 min.

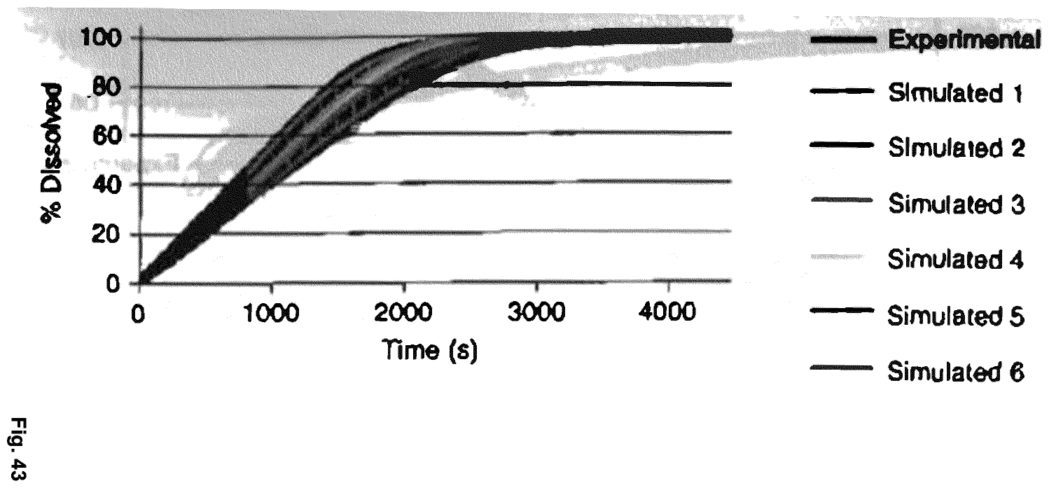

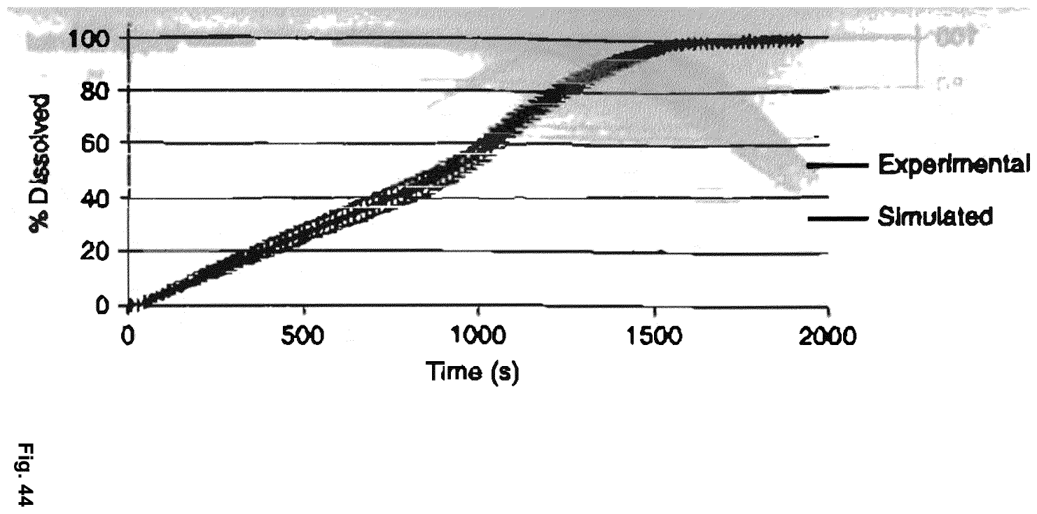

In comparison to models based on fitted equations, the CA-based approach significantly widens the technical possibilities of predicting, simulating, and modeling different phenomena occurring during the dissolution runs. As an example, the influence of the size of the granules on release profile 13 can be shown (see FIG. 43). The experimental release curves (line with error bars) were obtained by compacting 7 mm tablets of granulated caffeine (approximately 450 μm). In this respect, the deviation from the average curve is explained by random arrangement of the granules in the tablet, causing the fluctuations during the release. On the same FIG. 43, six simulated release curves are shown. The six virtual tablets were made to mimic the particles' size distribution of the real caffeine granulate. As can be seen, each simulated curve yields a different profile. This is because of the random arrangement of the granules each time the virtual tablet is constructed. It can be seen that the simulated curves very closely describe the variability of the experimentally acquired release profiles 12. In this particular example, the CA-based drug release generation is capable of root case finding for out-of-specification problems, i.e. formulation 13 induced release profile 12 variability. It is to be noted, that the present CA-simulation based system 1 can also be useful in the task of exploring various phenomena observed during the drug dissolution. One of such examples is presented in FIG. 44. In FIG. 44, the release curve 12 of the pure caffeine tablet without constraints, as generated by means of system 1, is illustrated. It can be seen that the release 12 rate at approximately 900 s is changing. This change is consistent for all six tablets dissolved, and yielded low variability as shown by the error bars. The visual inspection of the tablet behavior in the dissolution bath (USP 2) was possible throughout the whole dissolution. It has been observed that the tablets after 900 s of dissolution were falling apart, thus yielding a higher solid-liquid interface area. According to the Noyes-Whitney equation, such an event should increase the rate of the dissolution, which is successfully observed in FIG. 44. In order to prove this, the virtual tablets were constructed with higher porosity at the core of the tablet. When the outer, denser layer was completely dissolved, the inner part of the virtual tablet yielded a higher dissolution rare as its contact surface to the cells of type 200 (dissolution media, see the system's cell types, as discussed above) was increased. The resulting generated and simulated approximation is very close to the experimentally obtained release curve 12. It has to be kept in mind that the technical ability of the CA-based system 1 to generate and predict the release profile 12 features is only possible if the mechanism of the drug release from a tablet formulation 13 is understood. On the other hand, predicting, simulating and modeling with the present CA based system also allows to find the explanation for observed phenomena and to proof of the proposed explanation.

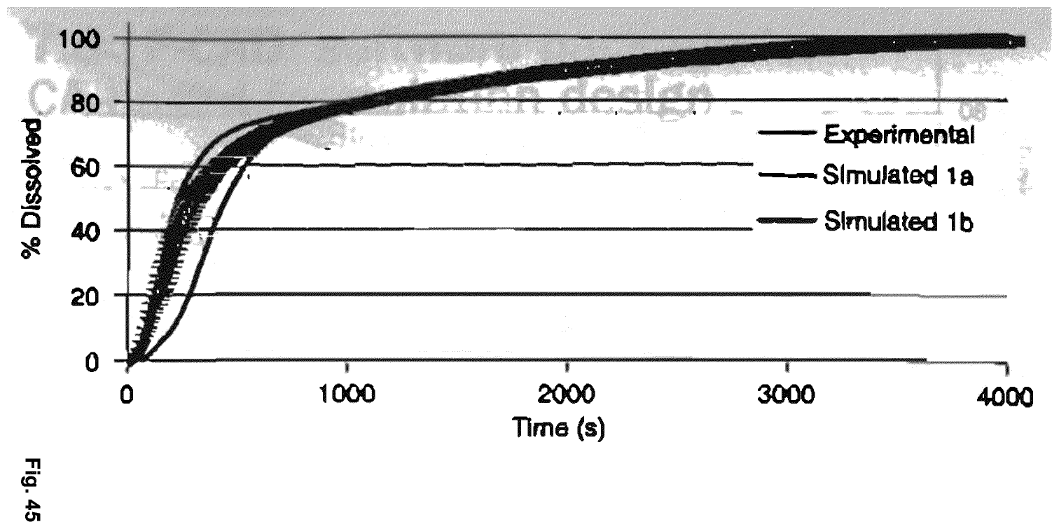

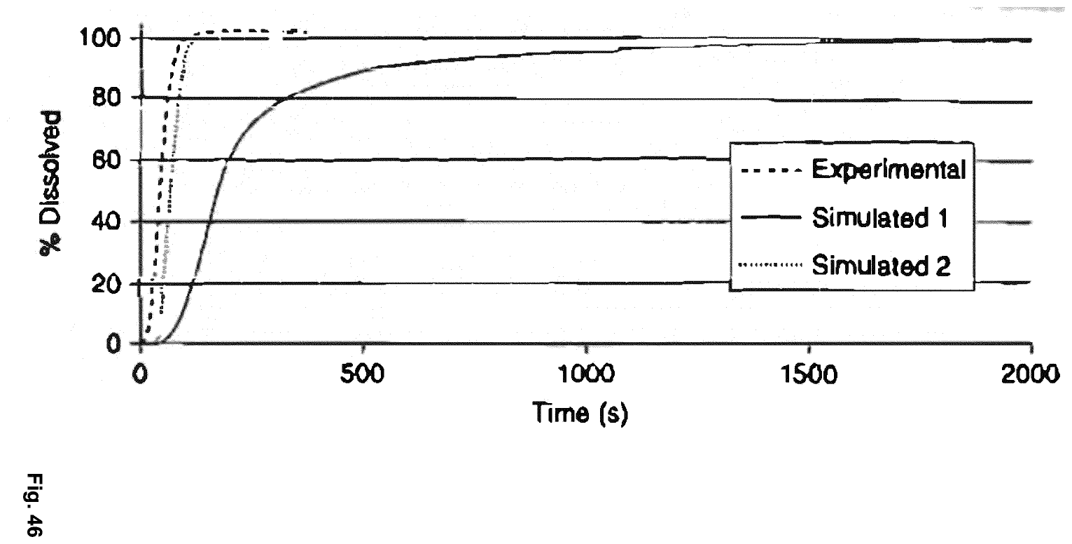

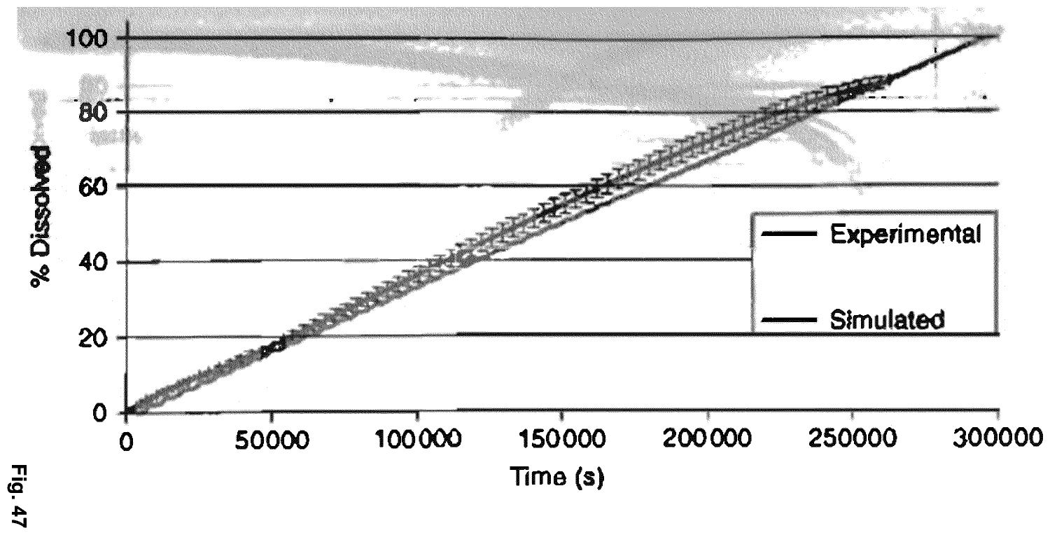

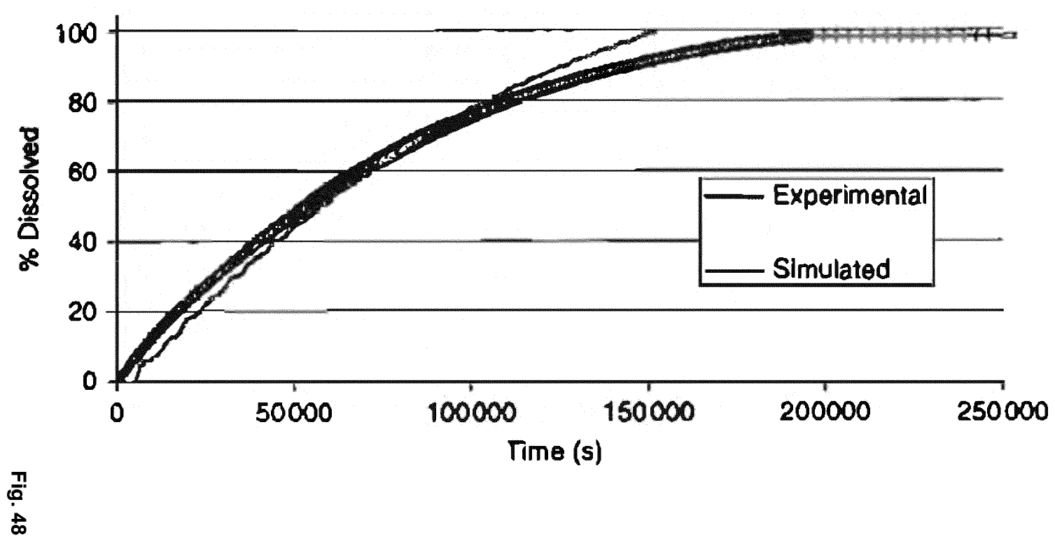

The few examples above concerned tablets compacted of pure API (caffeine). However, it is uncommon to have such formulations in reality for a multitude of reasons. In general, the tablet formulations contain 5-60% w/w of excipients which serve different functions: from compatibility and compressibility enhancement to release profile 12 modifications, etc. It is of a major interest to generate, calculate and study the influence of excipients on release rate of a tablet or capsule formulation 13. Further results of such a system 1 based processing are presented in FIG. 45, depicting the release profile 12 from caffeine tablet formulation with 54% v/v of MCC. In this formulation MCC plays the role of a disintegrant, forcing the caffeine crystals to expose a higher surface to the dissolution media. The simulated MCC function introduced such action to the vicinity of APJ cells. Similar to reality, such action forced an accelerated release from the CA-based tablet generation. The resulting curves (simulated 1 a and simulated 1 b) closely approximate the experimental release profile 12 of the tablet formulations 13 with MCC. In the next example, the disintegration action of MCC is enhanced by adding 2.5% v/v of crosscarmellose sodium (Ac-Di-Sol) to the formulation 133 with MCC (see FIG. 46). The synergistic effect of Ac-Di-Sol and MCC yields a higher release rate of caffeine as can be seen from FIG. 46. The imprecise simulation of the experimental release curve (simulated 1, synergistic effect of the disintegration is off) is shown for comparison with curve ‘simulated 2’, where the synergistic effect of the MCC and Ac-Di-Sol on the release rate is used. In this case, the synergy between chose two components was taken into account by reducing the primary constants for both excipients to 0, i.e. virtually ‘disintegrating’ the API cells in the vicinity of the excipient. The resulting simulation curve provides a very close approximation of the experimentally acquired release profile (see FIG. 46). Following the example with Ac-Di-Sol and MCC formulation, it must be kept in mind that successful simulation of the release profile 12 is not possible without understanding the physicochemical nature of the studied phenomena. The above-mentioned examples describe simulations of the formulations 13 with well-soluble drug substances. In the following example the low-soluble drug Proquazone is used in the example of generations and simulations for a more challenging formulation 13. The IDR profile of proquazone tablet is shown in FIG. 47. The simulated curve represents the virtual tablet simulated release profile with constrained geometry. The primary API constant was found and was kept for additional simulation of the release profile 12 where the virtual tablet was not subject to any geometry constraints, i.e. just a simulation of the pure API compacted tablet in dissolution media. As can be seen in FIG. 48, the simulated curve closely describes the experimentally obtained release curve, keeping the primary API constant intact. This example demonstrates that the influence of tablet geometry on the release rate of the tablet is significant, and this effect could be easily taken into account by the realized CA-based dissolution models. It is also important to highlight that the actual solubility of the API (caffeine or proquazone) does not influence the performance of the calculation model. As described above, the presented dissolution examples are generated by means of the CA-based formulation design, prediction and modeling system 1.

The present system 1 can be hardware- and/or partially software-based providing and supporting efficient and reliable tablet formulation generation and development. It is based on the technical concept of cellular automata, as described above. In this respect, the present system is not a classical expert system because it is not directly based on human expert knowledge but is a computer-aided, intelligent system, having its own intelligence and adaptivity. In accordance with the discussed details to the CA-based compaction and dissolution generations and model structures, the required input measuring parameters and data for system 1 are, due to its integrated intelligence and in contrast to the prior art systems, not extensive: only a few physicochemical properties of the compounds are needed. In fact, the input data can be split into two major parts: physicochemical and geometry-related parameters of pharmaceutical formulation 13. Physicochemical parameters describe components' solubility, water (or other solvent) sorption, swelling kinetics, and others. Geometry-related parameters span from individual component properties to granulate and tablet geometrical parameters, i.e. sizes, shape factors. Therefore, the system 1, by means of the technical structure used, is capable of taking into account solubility, swellability, and particle size distribution of alt compounds and any desired shape of a tablet. The system 1 can consist of several independent, functional modules or units (e.g. hardware- or software based). Further technical aims in developing the system 1 are: (i) Provide and enable an automated formulation design system with a minimum amount of drug substance and time; (ii) Explore formulations 13 which can technically not be tested in vitro due to lack of API or other reasons; (iii) Technically minimize laboratory experiments by the use of the present system 1, thereby, inter alia, also shortening time-to-market; (iv) Allow for a technical-based check for equivalence in case of excipient change; (v) Providing a technically controllable quality improvement; and (vi) Apply root-cause and technical-based measurement and analysis in case of ‘out of specification’ (OOS) problems.

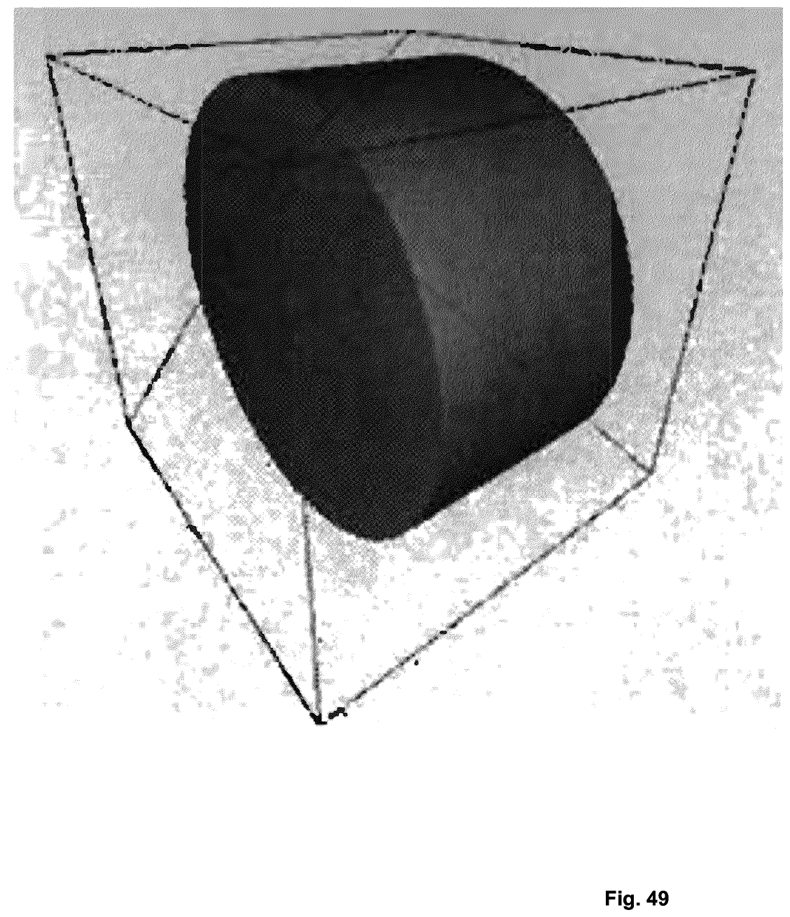

For constructional reasons, the system 1 can be described as consisting technically of several independent functional modules or units, all serving a different purpose during the automated design of a tablet. For an overview, the operational modules and engines 16/17/172/19 are listed in chronological order, as they can be exemplarily used during the general workflow of the system 1: (i) First task engine 16 (also referred as tablet designer (TD)) enabling tablet shape design with complex geometry. The output is a discrete tablet which is discretized and then transferred to the PAC (Particle Arrangement and Compaction) module 17; (ii) Discretizator module 172: This module enables transformation of the complex, arbitrary-shaped tablet into a discrete cubic grid (or 3-D matrix). This matrix serves as an input file for the second task engine 17; (iii) Second task engine 17 (Particle Arrangement and Compaction module): By this module 17, the virtual tablet is filled with particles and the complete composition of the formulation is entered. A rule-based growth structure is used to design different particle sizes and, in combination with seed distribution functionality, is enabled to mimic conditions after granulation; (iv) Dissolution module 19 with dissolution simulation (DS): To calculate the dissolution of a previously designed and packed tablet, the concept of cellular automata is used. The dissolution of every API cell is calculated using the generalized description of the solid-liquid interface which correlates to the Noyes-Whitney relation. In system 1, the virtual tablet can e.g. be designed using the described first task engine 16 (tablet designer). The first task engine can be realized in a way, that with this module 16, any arbitrary shape of tablet can be designed and further used with system 1. In addition, the system 1 generates and calculates the tablet volume and porosity if test compounds are filled into the matrix. The designed tablet is then transferred to the discretizer. In the discretizer module the matrix gets rendered. This process is based on a ray-casting discretization algorithm technics. Technically speaking, rays penetrate the cubic matrix in x- and y-directions. The distance between the penetrating rays correlate with the matrix side length, divided by the number given in ‘Cube size, units’. The ray case finds the coordinates of intersection points of the ray with the tablet shape. The matrix around the tablet is filled with unit cubes, except for the tablet, which forms a hollow space. After the process is done for the whole matrix, the matrix is inverted, finally producing a rendered tablet, filled with unit cubes. The number in ‘Cube size, units’ defines the number of cubes at the edges of the cubic grid around the tablet (FIG. 49). Like this, the size of the smallest unit (one particle) is specified. More cubes result in more cells which need to be calculated for every iteration of the dissolution generation and calculation process. Thus, it is not always possible to simulate the dissolution of a virtual tablet which contains as many particles as a real tablet. Consequently, there is a compromise to be made between precision of the processing, calculation and speed.

A possibly realized generalized task design and operation workflow for system 1 can e.g. consist of three main phases: 1. 3-D design of a tablet geometry in order to define the wall constraints; 2. packing the components into the hollow tablet; and 3. calibration for new API or direct calculation of the release profile 12, if calibration constants C1 and C2 were defined. In the case of a new active substance, calibration is mandatory. The calibration of an ingredient, API or an inactive excipient, is usually carried out with feedback to laboratory experiment measurements for intrinsic dissolution race (IDR) data. Attempts can be made to simulate the IDR profile with the system 1 using the dissolution constants at first approximation. Where the resulting release profile 12 deviates from the laboratory data, the optimization algorithm (Nelder-Mead simplex method) can e.g. be used to find the solubility constant at which the experimental and calculated release profiles 12 converge. The release profile 12 simulation is an iterative approach, i.e. the search for best and robust formulation 13 is performed in a circle until the best satisfactory result is achieved. It can further be also important to do confirmation trials and it can e.g. be necessary to recalibrate formulation excipients where there is low performance. Generally, the required data set for successful operation and development with the system 1 does not exceed the standard measuring parameters and data on new or generic API. The API-related data include solubility and stability profiles at different pH ranges, true density, particle size distribution. In the case of known polymorphs, the same set of data should be readily available for different polymorphic modifications. For calibration purposes the intrinsic dissolution rates for an API of interest are required. The excipients used muse be investigated also for their particle sizes, true densities and any effect on the API solubility. The latter is usually accomplished by studying the IDR of binary mixtures between an exc1p1ent and an API of interest. For simplicity, the IDR of a trial formulation consisting of the API and of excipients of interest, which need to be chemically compatible, is often tested at first. It is important to note, that once being characterized an excipient can be further used with different APIs and excipient mixtures.

As an embodiment variant, the invention e.g. provides automated compliance with compliance parameters such as colors, shapes etc. In this embodiment variant, a TabletDesigner module can be used to develop the custom shapes and volumes of the final medicinal product. This includes, inter alia, the automated handling and triggering of parameters which are compatible with human physiology and are allowed by country or region-specific guidelines. Further limitations on the tablet geometries include shape compatibilities with the production equipment, such as the maximal or minimal size of a tablet. The TabletDesigner module monitors users' activities and corrects the errors online during the design of the shape and volume of the tablet. The tablet design process is split into two states: 1) the 2-dimensional design user interface and 2) the 3-dimensional user interface. In the 2D designer mode, the user, e.g. can be requested to draw a die shape of the product and select the height, caps design, and breaking notch. Every tablet design element has the fundamental limitations dictated by the manufacturability-assessment heuristics programmed in. As an example, a heuristic rule can comprise: IF tablet diameter OR tablet dimensions are greater than 20 mm, THEN the tablet cannot be manufactured by standard rotary tablet press THUS warn the user. In the 3-dimensional modus of operation, the user can generate the final 3d shape of the tablet or a capsule, and the total surface, height, and volume will be calculated and displayed by the software. In addition, the user may select the color of the tablet by applying the region or application-specific color schemes available. The color palettes are designed to contain color allowed for use in humans and are non-toxic. In some territories, specific colors are limited due to religious, economic, or psychological limitations. For example, the medicinal product cannot feature an attractive shape or colors to prevent their misuse by children. Further, the TabletDesigner module can contain heuristics to guide the user and prevent the design of the medicinal product, which cannot be manufactured or used in a specific region or a defined field of application.

As a further advantage of the present invention, the invention is able to provide an automated connection between laboratory and simulation for excipients as well as application programming interfaces (API) using a interface to excipient manufacturers. In this embodiment variant, a material database connector unit interfaces can e.g. provide the connectivity of a F-CAD internal material database with the material producer. The connector's functionality can e.g. include an accessibility interface to the non-SQL document-oriented online database. The material characteristics can e.g. be stored in the JavaScript Object Notation (JSON) object format, i.e., they do not have the limitations of the standard relational databases. The limits are seen as the unavailability of a particular predefined product characteristic, and the supplier may omit the publication of this property in the database. The latter will limit the simulation performance of this material during the in-silico product design; however, it will still allow this component in the simulations. On the contrary, if the manufacturer of the excipient or an active pharmaceutical ingredient has different properties that do not have predesigned placeholders in the document structure, the introduction of those will not cause a system malfunction; those properties will be made available to the end-user of the software. If subsequent versions of the simulation make use of this additional information, then they are already available. The material document can further comprise means for accepting dynamic data, e.g., solubility vs. pH or viscosity vs. concentration. Typically, information provider supplies only measured selected data points; the rest of the inter and extrapolation will be carried out by the F-CAD simulation software using the cubic spline interpolation. An example of the JSON format of the database record for a selected material can e.g. be as follows:

```
{ “uid”:1,
        “name”:“Theophilline”,
        “producer”:“CINCAP”,
        “type”:1,
        “amount”:0,
        “initialAmount”: 0,
        “density”:1453,
        “actualcells”:0,
        “wfraction”:0,
        “vfraction”:0,
        “solubility”:[{“key”:7, “value”:7.36}],
        “molarMass”:180.164,
        “viscosity”:0,
        “viscosityAlpha” : 0,
        “capillaryConstant”:0,
        “contactAngle”: 0,
        “waterBindingRate”:1,
        “LOD”:0.00,
        “diffusivity”:6.16e−10,
        “tension” : 0.0,
        “ssa”:0.1,
        “psize”:0.050,
        “poreSize” : 1.6e−3,
        “porosity”: 0,
        “formFactor”:1,
        “color”:“#ca1f48”
}
```

As another advantage of the present invention, the invention is able to provide an automated connection between lab and simulation for machine process types as well as APIs e.g. for Interface to Machine Manufacturers. The characteristic of the solid pharmaceutical formulations, such as disintegration time, release rate, hardness, etc., depend not only on the composition of the formulation but also on the order and process parameters when constituents were put together. The results may differ tremendously depending on the apparatus model and mode of operation for the same process, e.g., for wet granulation or compaction. The used F-CAD simulation platform offers a built-in compiler language to automate the production of the virtual tablets or granules according to the trait of used machines or processes. For example, the granules in different devices will have different porosity of the granules or roundness of the resulting particles. These changes are introduced by different impeller or chopper geometries and the shape of the granulation vessels or allowed working temperatures. With the help of the built-in programming language, it is possible to exactly match the typical unit operations' behaviors. For example, the invention allows to automatically specify the granulation's typical granulometric composition and final granules' porosity for different constituents to match the specific granulator model. This process requires data obtained by the equipment manufacturer for standard formulations and generalized to other components with the help of the language lexemes. An example of script language can be given as follows:

```
program FCADTEST;
var
  filename,emsg:string;
  ha: TStrings;
  i: integer;
begin
  filename := ‘test.3dpac2’;
  try
    CreateMatrix(154,154,154,0.05);
    MakeRoundTablet(100,50);
    AddComponent(1, ‘ScriptAPI’, 1.2345, clRed);
    Distribute(1, 5, 20);
    Seed(1);
    Grow(1);
    Grow(1);
    Grow(1);
    UpdateStatistics;
    ha := getComponentHistogram(1);
    Writes(‘Length:’);Writel(ha.count);WriteIn;
    for i := 0 to ha.count-1 do begin
    Writes(ha[i]);
    end;
    SaveMatrix(filename);
  except
    emsg:=ExceptionToString(ExceptionType, ExceptionParam);
    writes(emsg);
    //do somethign with the exception message i.e. email it or
    //save to a log etc.
  end;
end.
```

Regarding scale up (Buckingham Theorem) and dimensionless parameters, the above-mentioned interface to machine manufacturers can e.g. use the language developed for F-CAD to build a simulation model of the typical behavior of the specified machine and to generalize this behavior on the other components of the formulations. These models can be chained or applied in parallel to come as close as possible to describe the physical process. The mathematical models are based upon dimensionless characteristic numbers or criteria. These numbers allow describing the complex physical or physical-chemical phenomena with relatively simple criteria-based equations with exponents. For example, the Navier-Stokes equation can be represented in the criterial form as follows:

$$\frac{Δ p}{ρ {v}^{2}} = {( \frac{vd ρ}{μ} )}^{m} {( \frac{{v}^{2}}{gd} )}^{n} {( \frac{vt}{d} )}^{p} {( \frac{l}{{d}_{e}} )}^{q}$$

The use of criteria such as Reynolds or Nusselt assumes that those values, once obtained for the laboratory scale experiment, will be valid for further investigations at a larger scale, in theory, at an infinitely large scale. This concept allows for scaling up or down of the descriptive process models used by the F-CAD for simulation of the medicinal product properties at any given scale. The models are built-in with the help of the F-CAD programming language.

In an embodiment variant, the invention acts as an ERP solution for the whole product which allows integration into external manufacturing/supply chain solutions. In this embodiment variant, the TabletDesigner module, Interface to Excipient Manufacturers, Interface to Machine Manufacturers, and Scale-Up are considered as a daisy chain model piggybacking on the F-CAD simulation engine based on the cellular automata (a form of the liner approximations-based numerical method to solve systems of the PDEs) the full functionality of the system is represented in the form of an ERP solution. The F-CAD system is a cloud-based integrator platform offering a user interface to the database access based on different presentations, such as Machine Manufacturers Interface, Excipients Manufacturers Interface, or simulation platform itself, including but not limited to TabletDesigner, Particle Arrangement Module, and the Dissolution Simulations Module. This platform allows Participants in the Pharmaceutical Development, Production and Distribution Value Chain to interact, integrate and orchestrate with other Participants according to their Business Model, either on an End-to-End or Peer-to-Peer basis.

### LIST OF REFERENCE SIGNS

1 Automated formulation design system
   11 Feed Components
      111 Active pharmaceutical ingredient (API)
         1111 Material Attributes
         1112 Material Attribute Functional Coefficients
         11121 Floating Point Functional Coefficients
         11122 Integer Functional Coefficients
         11123 Short Word Functional Coefficients
         11124 Byte Type Functional Coefficients
      112 Excipients
         1121 Material attributes
         1122 Material Attribute Functional Coefficients
         11221 Floating Point Functional Coefficients
         11222 Integer Functional Coefficients
         11223 Short Word Functional Coefficients
         11224 Byte Type Functional Coefficients
   12 Release Profile
      121 Body fluid
         1211 Interstitial fluid
         1212 Intracellular fluid
      122 Desired/Targeted release profile
         1221 Targeted dissolution factor
         1222 Targeted solubility factor
         1223 Targeted protection of the API
      123 Measured/Simulated release profile
         1231 Measured dissolution factor
         1232 Measured solubility factor
         1233 Measured protection of the API
   13 Tablet/Capsulation Formulation
      131 Components of Tablet/Capsulation
         1311 API Components
         13111 Shape
         13112 Size
         13113 Configuration
         13114 Quantity
         1312 Excipient Components
         13121 Shape
         13122 Size
         13123 Configuration
         13124 Quantity
      132 Structural Elements
         1321 Granule
         1322 Layer
         1323 Coatings
         1324 Shape
   14 Physical Effect
      141 Measured Effect
      142 Desired Effect
   15 Calibration Module
      151 Simulation Environment
      152 Real World Environment
   16 First task engine (tablet designer)
      161 Targeted physical configuration
   17 Second task engine (also referred as PAC module)
      171 Operational Configuration
         1711 Process
         1712 Machines
         1713 Operating parameter
      172 Discretizator module
   18 Polyhedral meshwork structure
      181 Discretized voxels
      182 Dimensions of the meshwork structure
      183 Face
      184 Edge
      185 Vertex
   19 Dissolution module
      191 Diffusion Simulation
      192 Dissolution Simulation
      193 Disintegration Simulations
      194 Swelling Simulation
      195 Compression Simulation
      196 Post-Compressive Stress Distribution and Lamination/Capping Simulation
      197 Deformation simulation

1. Automated formulation design system for determining tablet and capsulation formulations providing a targeted delivery of active pharmaceutical ingredients to a patient with a desired release profile in defined body fluids based on experimental measurements of material attributes for simulated dissolution, disintegration, diffusion, compression, deformation and/or other properties of individual materials or combinations and mixtures thereof, wherein the formulation at least comprises required quantities of the active pharmaceutical ingredients and excipients providing targeted dissolution and/or solubility factors and/or targeted protection of the active pharmaceutical ingredients during their transport and distribution into interstitial and intracellular fluids characterized,
in that system comprises a first and second task engine, the first task engine capturing a targeted physical configuration of a tablet or capsule as end product comprising at least a shape and/or size and/or configuration of the active pharmaceutical ingredients and/or excipients into elements comprising granules, layers and/or coatings, and the second task engine determining processes, machines and/or operating parameters required to achieve the targeted physical configuration at a laboratory scale clinical trials and/or at large scale for mass production,
in that the system comprises discretized voxels in from of a polyhedral meshwork structure, wherein a combination of measured material attributes is mappable onto the discretized voxels of the polyhedral meshwork structure, wherein the polyhedral meshwork structure is defined in an adaptable number of dimensions, each representing an individual material, when mapped onto a face or a vertex of the meshwork polyhedron, and
in that the system comprises a dissolution module simulating the production of the end product by varying the targeted physical configuration of the first task engine, and the parameterized processes and machines and operational parameters of the second task engine, and by generating the effect of variances in component, process and machine and operating parameters and the corresponding release profile of the tablet or capsule as end product.

2. The automated formulation design system for determining tablet and capsulation formulations according to claim 1, wherein said individual materials are represented by means of floating point material attribute functional coefficients, when mapped onto each face or an edge or a vertex of a meshwork polyhedron.

3. The automated formulation design system for determining tablet and capsulation formulations according to claim 1, wherein said individual materials are represented by means of integer functional coefficients, when mapped onto each face or an edge or a vertex of a meshwork polyhedron.

4. The automated formulation design system for determining tablet and capsulation formulations according to claim 1, wherein said individual materials are represented by means of short word functional coefficients, when mapped onto each face or an edge or a vertex of a meshwork polyhedron.

5. The automated formulation design system for determining tablet and capsulation formulations according to claim 1, wherein said individual materials are represented by means of byte type functional coefficients, when mapped onto each face or an edge or a vertex of a meshwork polyhedron.

6. The automated formulation design system for determining tablet and capsulation formulations according to claim 1, wherein for said combination all material attributes from the simulated mixture or mixtures thereof are storable by means of the voxel.

7. The automated formulation design system for determining tablet and capsulation formulations according to claim 1, wherein the combinations are usable in diffusion simulation.

8. The automated formulation design system for determining tablet and capsulation formulations according to claim 1, wherein the combinations are usable in dissolution simulation.

9. The automated formulation design system for determining tablet and capsulation formulations according to claim 1, wherein the combinations are usable in disintegration simulations.

10. The automated formulation design system for determining tablet and capsulation formulations according to claim 1, wherein the combinations are usable in swelling simulation.

11. The automated formulation design system for determining tablet and capsulation formulations according to claim 1, wherein the combinations are usable in compression simulation.

12. The automated formulation design system for determining tablet and capsulation formulations according to claim 1, wherein the combinations are usable in post-compressive stress distribution and lamination/capping simulation.

13. The automated formulation design system for determining tablet and capsulation formulations according to claim 1, wherein for the simulation and development of a targeted tablet or capsule formulation achieving a targeted release profile in simulated body fluids, the system comprises a calibration module calibrating the simulation environment and the system to match real-world behavior of substances and processes, wherein the formulation is developed by means of the calibrated simulation environment.
